(19) United States
(12) Patent Application Publication
(10) Pub. No.: US 2024/0335829 A1
(43) Pub. Date: Oct. 10, 2024

(54) SAMPLE COLLECTION DEVICES

(71) Applicant: ConceptoMed AS, Ballstad (NO)

(72) Inventors: Christian Mide, Ballstad (NO); Jimmy Gido Schon, Ballstad (NO)

(21) Appl. No.: 18/294,850

(22) PCT Filed: Aug. 2, 2022
(86) PCT No.: PCT/EP2022/071765

§ 371 Date: Feb. 2, 2024

(30) Foreign Application Priority Data
Aug. 2, 2021 (GB) 2111128.1
Mar. 28, 2022 (GB) 2204390.5

(51) Int. Cl. B01L 3/00; A61B 10/00

(52) U.S. Cl. CPC B01L 3/502; A61B 10/0051; B01L 2200/026; B01L 2200/0689; B01L 2300/042; B01L 2400/0478; B01L 2400/06

(57) ABSTRACT

A sample collection device for collecting a fluid sample, including a sample collection chamber for collecting a sample therein, wherein the chamber includes a chamber outlet through which a fluid sample can pass. The device further includes a sample collection conduit extending through an opening of sample collection chamber to an outside of the sample collection chamber, wherein the sample collection conduit includes a conduit inlet for receiving a fluid sample and a conduit outlet for allowing the fluid sample to pass into the sample collection chamber. The device further includes a plunger configured to expel the fluid out of the sample collection chamber through the chamber outlet.

The present invention relates to sample collection devices for use in collecting fluid samples, for example oral fluid samples.

Oral fluid samples, for example fluid samples comprising human saliva or sputum, can be analysed to determine the makeup of the sample. In recent developments, human saliva may be used to determine whether a provider of the sample is infected with a virus, for example severe acute respiratory syndrome coronavirus 2 (SARS-COV-2) which may indicate that the provider of the sample has coronavirus disease (COVID-19). The testing of oral fluid samples to determine whether a provider of the sample has SARS-COV-2 may provide an improved means for testing for the virus when compared to other prior art techniques. The same may be true for other viruses or bacteria, e.g. influenza, respiratory syncytial virus, hepatitis virus or tuberculosis. Prior art techniques for testing include the use of swabs, which are relatively invasive and can in some instances be uncomfortable for the provider of the sample.

The analysis of an oral fluid sample typically requires a relatively precise amount of sample for reliable analysis. In order to acquire a precise volume of sample, prior art techniques typically involve the use of a saliva sample kit. Such a kit often includes a number of parts, for example seven different separate components, for use in collecting and measuring a sample. Such kits normally require a user to spit or drool into a funnel which is attached to a measurement vial. The measured saliva is then poured into a secondary vial, and a lid is sealed onto both the measurement vial and the secondary vial. Use of these prior art kits presents a significant risk of contamination of a sample, particularly as the sample is transferred from one container to the other. Further, in the act spitting or drooling into the measurement vial, and subsequent transferring of the sample to the secondary vial, there is a significant risk of inadvertently leaking the sample onto vial threads and other surfaces, such as a work surface, or a user's hands. As the sample may be infected, this therefore increases the risk of infecting others which is clearly not ideal. Additionally, it has been found that spitting into a measurement vial may produce aerosols which pose a risk of airborne contamination of other individuals in the vicinity of the person delivering the sample. Furthermore, the amount of oral fluid has been shown to vary substantially when collected by the individual on its own, affecting reproducibility and confidence in the results of such analyses.

Accordingly, while efforts have been made to collect and measure an oral fluid sample for analysis, the prior art devices and techniques clearly have significant limitations and pose potential risks.

The present invention aims to address, or at least mitigate, one or more of the problems outlined above and when viewed from a first aspect provides a sample collection device, for collecting a fluid sample, comprising:
a sample collection chamber for collecting a sample therein, wherein the chamber comprises a chamber outlet through which a fluid sample can pass;
a sample collection conduit extending through an opening of sample collection chamber to an outside of the sample collection chamber, wherein the sample collection conduit comprises a conduit inlet for receiving a fluid sample and a conduit outlet for allowing the fluid sample to pass into the sample collection chamber; and
a plunger configured to expel the fluid out of the sample collection chamber through the chamber outlet.

Accordingly, as will be appreciated by those skilled in the art, through the use of the sample collection conduit, a user may collect their own fluid sample without requiring assistance from others, e.g. healthcare personnel. The fluid sample collection device may be suitable for collecting any fluid sample, however the sample collection device may be used for collecting an oral fluid sample, e.g. comprising saliva or sputum. Given that the sample may, in some instances, be infected with bacteria or a virus, by collecting the sample themselves, the chance of healthcare personnel coming into contact with the potentially infected sample is minimized. By reducing the number of interactions of healthcare personnel with potentially infected persons, this may also reduce the consumption of personal protective equipment (PPE), such as gloves, masks, aprons, etc, which healthcare personnel often have to change frequently, when collecting samples. Further, the lack of need for assistance during sampling and further processing may also substantially reduce costs and resources, e.g. of healthcare personnel, associated with collecting and analysing samples. The opening of the sample collection chamber may be at, or considered to be, a first end of the sample collection chamber and the chamber outlet may be at, or considered to be, a second end of the sample collection chamber. In extending through an opening of the sample collection chamber, a first portion of the sample collection conduit may extend from within the sample collection chamber such that the conduit inlet is outside of the sample collection chamber. In at least this position, the sample collection conduit may extend from the sample collection chamber such that a user is able place their mouth around the sample collection conduit so as to provide a sample through the sample collection conduit.

Further, through the use of a sample collection conduit which conveys the sample directly into the sample collection chamber, the sample may be kept isolated from the environment thereby minimizing the chance of the sample becoming contaminated. The sample may pass directly from a user's mouth, into the sample collection conduit, and into the sample collection chamber without being exposed to the external environment. This may help to ensure that any analysis performed on the sample accurately reflects the sample of the provider, without contamination from others. Additionally, by collecting the sample using the sample collection conduit, the provider of the sample is less likely to get the sample over their hands, which reduces the likelihood of the provider going on to inadvertently deposit their sample on other surfaces. Furthermore, collection of the fluid sample using the sample collection conduit may reduce the risk of the formation of aerosols in the supplying of the fluid sample, e.g. via spitting. This may therefore reduce the risk of airborne contamination of others in the vicinity, or indeed work surfaces in the vicinity.

Use of the device may involve a user placing their mouth around the conduit inlet. The user may then transfer a sample, e.g. comprising saliva, through the sample collection conduit into the sample collection chamber. Transferring of the sample may be achieved by the user applying a spitting action into the sample collection conduit. This may have to be repeated a plurality of times, often up to and above 10 times, to achieve the sufficient volume. Once the sample collection chamber contains a sufficient volume of the sample, the sample may be controllably expelled from the sample collection chamber using the plunger. This may be performed immediately after providing the sample, or the sample may be temporarily stored within the sample collection chamber before it is expelled. The sample may be expelled into or onto in any suitable means, e.g. a means for testing. As will be appreciated, fluid such as the sample, or air subsequently blown by the user to flush the sample collection conduit of any remaining sample therein, may pass through the sample collection conduit into the sample collection chamber.

The plunger and the sample collection conduit may be provided by separate parts which may be free to move within the sample collection chamber independently. In a set of embodiments, the sample collection conduit is moveable relative to the sample collection chamber and wherein the sample collection conduit and plunger are operatively linked such that movement of the sample collection conduit causes movement of the plunger within the sample collection chamber. Operatively linking, i.e. coupling, the movement of sample collection conduit and the plunger in this manner may advantageously mean that the sample collection conduit can be used to drive movement of the plunger within the sample collection chamber. This may provide a convenient means for operation of the device. The operative link may be achieved in any suitable manner. For example, the sample collection conduit may act upon the plunger, in order to drive its movement. In a set of embodiments, however, the sample collection conduit and the plunger are integrally formed. For example, the plunger may be formed in, or on, a wall of the sample collection conduit. This wall could be a base wall or a side wall of the sample collection conduit. For example, the plunger may surround the sample collection conduit, which may be cylindrical. The plunger may be arranged part way along the length of the sample collection conduit such that the sample collection conduit extends above and below the plunger. The sample collection conduit and the plunger may be integrally formed as a single component by any suitable means, for example by integrally forming the parts together in a single mould. In such an embodiment, the movement of the plunger will be inherently coupled to the movement of the sample collection conduit. By integrally forming the sample collection conduit and plunger as a single part, the number of separately operable components on the device may be reduced, and thus the use of the device may be simplified for a user. Further, the manufacture may be simplified, and the cost of manufacture may also be reduced.

In embodiments wherein the movement of the sample collection conduit and plunger is operatively linked, i.e. coupled, the sample collection conduit may be configured to be pushed into the device by a user. This may, for example, comprise the user pushing the sample collection conduit into the sample collection device using their hand, e.g. a thumb, or by pushing the sample collection conduit against another surface. This may therefore advance the sample collection conduit and the plunger into the sample collection chamber. Alternatively, the sample collection conduit may be configured to be rotated in order to advance the sample collection conduit and plunger into the device. For example, the sample collection conduit, or a part thereof such as the plunger, may comprise a threaded portion configured to engage with a corresponding threaded portion on the sample collection chamber. Accordingly, in such embodiments, rotation of the sample collection conduit relative to the sample collection chamber results in the sample collection conduit and plunger advancing into the sample collection chamber. Such an embodiment may allow for more control over the advancement of the plunger and thus more control over the expelling of the sample from the sample collection chamber.

As discussed above, the sample collection conduit and plunger may be operative linked such that their movement is coupled. In a further set of embodiments, the sample collection conduit is movable from an initial position, whereby the sample collection conduit extends out of the sample collection chamber to receive a fluid sample, to a final position whereby the sample collection conduit is fully advanced into the sample collection chamber. As will be appreciated, during at least part of the movement from the initial position to the final position, the movement of the sample collection conduit will cause the plunger to expel the fluid sample from the sample collection chamber via the chamber outlet. In the final position, the sample collection conduit may be fully contained within the sample collection chamber such that it no longer extends to an outside of the sample collection device. As a result, once in the final position, the user may no longer be able to use the sample collection conduit as a means for providing a fluid sample. Of course in some embodiments, the sample collection conduit may still protrude out of the sample collection chamber when in the final position.

The sample collection chamber may have any suitable form that is capable of housing the fluid sample. The sample collection chamber may, for example, be substantially cylindrical in shape and be defined by an outer cylindrical wall. In a set of embodiments, the sample collection chamber comprises a first sub-chamber defined between the plunger and the chamber outlet, and wherein the conduit outlet is arranged in the first sub-chamber. In such a set of embodiments, when a fluid sample is provided via the sample collection conduit, the fluid sample will pass into and fill the first sub-chamber. The fluid sample may thus be at least partially contained within the first sub-chamber. In a further set of embodiments, the sample collection chamber comprises a first fluid passageway arranged to allow fluid to pass out of the first sub-chamber. The first fluid passageway may thus advantageously allow fluid to pass out of the first sub-chamber. The first fluid passageway may allow fluid to pass into another portion of the sample collection chamber, e.g. a second sub-chamber as will be described in more detail below. The first fluid passageway may be arranged such that it is open so as to permit the passage of fluid therethrough when the sample collection conduit is in the initial position, and thus when the plunger is a corresponding initial position. The plunger and first fluid passageway may be configured such that when the plunger is in the initial position, the first fluid passageway is open. The presence of such a fluid passageway may advantageously allow excess fluid to pass out of the first sub-chamber as the first sub-chamber is filled with a fluid sample. In the exemplary case of a fluid sample in the form of saliva, the Applicant has recognised that the first fluid passageway may advantageously allow any foam within the saliva sample to pass out of the first sub-chamber via the first fluid passageway. The first fluid passageway may therefore facilitate the separation of fluids within the fluid sample, e.g. the separation of the foam and the liquid part of a saliva sample. In the case of saliva, this may result in the fluid sample within the first sub-chamber being mostly liquid.

In another set of embodiments, the sample collection device comprises a further plunger arranged within the sample collection chamber, and wherein a second sub-chamber is defined between the plunger and further plunger. In such embodiments, the first fluid passageway may fluidly connect the first sub-chamber and second sub-chamber, for example at least when the sample collection conduit is in the initial position. Any excess fluid within the first sub-chamber may therefore flow out via the first fluid passageway into the second sub-chamber. The second chamber which is delimited by the further plunger may advantageously help to prevent any fluid sample from spilling out of the sample collection chamber. Similarly to the plunger described above, the sample collection conduit and further plunger may be operatively linked such that movement of the sample collection conduit causes movement of the further plunger within the sample collection chamber. The further plunger may be provided in any suitable manner, as described above with respect to the plunger. For example, the further plunger may be integrally provided with the sample collection conduit.

In a further set of embodiments, the further plunger is configured to move together with the plunger, and wherein the sample collection chamber comprises a second fluid passageway arranged to allow fluid to pass out of the second sub-chamber. The second fluid passageway may be open, i.e. to allow the passage of fluid therethrough, when the sample collection conduit, and thereby the further plunger, is in the initial position. The second fluid passageway may advantageously permit the escape of fluid from the second sub-chamber to another portion of the sample collection device, e.g. a third sub-chamber defined between the second plunger and a cap (where provided), as discussed in further detail below. The device may comprise another plunger, i.e. a third plunger, arranged within the sample collection chamber such that the third sub-chamber is defined between the further plunger and the third plunger. The presence of another plunger closing the third sub-chamber may further reduce the risk of the fluid sample escaping the device. Of course any number of plungers and sub-chambers may be provided. Allowing fluid to escape the second sub-chamber may further facilitate the escape of fluid from the first sub-chamber which may allow any excess fluid or foam to escape the first sub-chamber more easily.

The sample collection conduit may be moved out of the initial position by advancing the sample collection conduit into the sample collection chamber. In a set of embodiments, the plunger is movable to a first intermediate position in which the first fluid passageway is closed. In embodiments whereby the plunger is operatively linked to the sample collection conduit, this may be achieved by moving the sample collection conduit into the initial position. The plunger may act to close the first fluid passageway in any suitable manner. For example, the plunger may act on a part, e.g. a valve, which acts to close the first fluid passageway. Alternatively, the first fluid passageway may be in the form of a recess in a side wall of the sample collection chamber which allows the fluid sample to bypass the plunger when the plunger is adjacent the first fluid passageway. As will be appreciated by those skilled in the art, in such examples, when the plunger is no longer adjacent the first fluid passageway, the fluid will no longer be able to bypass the plunger and the first fluid passageway will effectively be closed. Closing of the first fluid passageway when in the first intermediate position may seal the first sub-chamber and thereby securely contain any fluid sample therein.

In a further set of embodiments, the further plunger may be moved into a corresponding first intermediate position whereby the second fluid passageway is also closed. This may be achieved by moving the sample collection conduit into the corresponding first intermediate position. In this further set of embodiments, closing of the second fluid passageway may thereby seal the second sub-chamber, and thus securely contain any fluid sample within the second sub-chamber. The further plunger may close the second fluid passageway in the same manner as described above with respect to the plunger. Closing the first and second sub-chambers may advantageously act to seal the fluid sample within the sample collection device. As a result, the fluid sample may be stored within the sample collection device and transported within the sample collection device without risk of the sample leaking. Use of the sample collection conduit, together with the plunger and further plunger as a means for securely closing the first and second fluid passageways may provide a convenient means for sealing the first and second sub-chambers.

The first sub-chamber may be dimensioned such that the volume of the first sub-chamber corresponds to a volume of fluid sample required for performing further analysis. As such, when the first sub-chamber has been filled, the entire volume of the first sub-chamber may then be dispensed out of the chamber outlet. This may effectively leave the device, i.e. the first sub-chamber, empty of the fluid sample. The position of first fluid passageway within the sample collection chamber may define the volume of fluid which is dispensed by the plunger. However, the Applicant has recognised that particularly with fluid samples such as saliva, even when fluid is able to escape the first sub-chamber via the first fluid passageway, some foam and air bubbles may nonetheless remain in the first sub-chamber. Such foam and air bubbles reduce the amount of liquid sample which is within the first sub-chamber.

For some types of sample fluid and particular types of analysis, a specific volume of liquid sample is required in order to perform meaningful analysis. Accordingly, in a further set of embodiments, the sample collection chamber comprises a third fluid passageway arranged such that when the plunger is moved into a second intermediate position, between the first intermediate position and the final position, the third fluid passageway is open, thereby allowing fluid to pass from the first chamber into the secondary chamber, and wherein the third fluid passageway is closed when the plunger is moved into a third intermediate position between the second intermediate position and the final position. The plunger may be moved between these positions by moving the sample collection conduit into and between corresponding second and third intermediate positions. Accordingly, as will be appreciated by those skilled in the art, the third fluid passageway therefore may act to define a final volume of fluid within the first sub-chamber such that a fixed volume of fluid can be dispensed from the device. As the plunger is advanced between the second intermediate position and the third intermediate position, the third fluid passageway is open and fluid is able to escape the first sub-chamber. As the plunger may advance with the sample collection conduit, fluid within the first sub-chamber will be forced out of the first sub-chamber via the sample third fluid passageway. This may advantageously force any remaining foam and air bubbles, or at least a portion thereof, out of the first sub-chamber. As a result, the liquid content of the fluid sample within the sample collection chamber may be increased.

In a further set of embodiments, the sample collection device comprises a fourth fluid passageway arranged to allow fluid to pass out of the second sub-chamber when the sample collection conduit is in the third intermediate position. The fourth fluid passageway may facilitate the flow of fluid out of the second sub-chamber, thereby allowing fluid to flow into the second sub-chamber from the first sub-chamber more easily. In a set of embodiments, the third and fourth fluid passageways are arranged such that they are closed simultaneously, when the sample collection conduit reaches the third intermediate position. This may advantageously mitigate the risk of leakage from sample collection device.

The first, second, third and/or fourth fluid passageways may have any suitable form which allows fluid to pass between the sub-chambers, and which can be appropriately closed, e.g. by the plunger or further plunger. In a set of embodiments, the first, second, third and/or fourth fluid passageway each comprise at least one recess in an internal wall of the sample collection chamber. The recess may be integrally formed as part of a moulding process used to manufacture the device. The use of a recess in an internal wall within the sample collection chamber may provide a simple and easy to manufacture fluid passageway within the device. In such embodiments, the first, second, third or fourth fluid passageway will be open, i.e. to allow the passage of fluid, when the respective plunger is adjacent the recess, and closed when the plunger is not adjacent the recess.

The sample collection device may allow a user to measure the volume of sample provided. As discussed above, this may be important, as certain types of analysis require a specific volume of sample in order for the analysis to function properly and produce accurate results. Measurement of the sample may be achieved in any suitable manner. For example, the sample collection chamber, e.g. the first sub-chamber, may be dimensioned such that when it is full it contains the correct amount of sample required for optimal analysis. The sample collection chamber may thus be a sample measurement chamber. Accordingly, in this case, the user may simply provide their sample until the sample collection chamber is full, and then stop. The sample collection chamber, and indeed other parts of the device, may be made from a transparent material such that the user can see the sample within the device. In the exemplary case of testing for SARS-COV-2, the first sub-chamber may have a volume of 1 ml. In embodiments comprising a third fluid passageway, the third fluid passageway may define a volume within the first sub-chamber of 1 ml. Of course the volume of the sample collection chamber, or the first sub-chamber may be dependent on the type of sample the device is intended for use with. The sample collection chamber may, for example, have a volume of between 1-20 ml, e.g. between 2-15 ml, e.g. between 3-8 ml, e.g. 7 ml. The volume of the sub-chambers within the sample collection chamber may depend on the volume of the sample collection chamber. In a set of examples, the first sub-chamber may have a volume of between 0.5 ml and 1.5 ml.

In another set of embodiments, the sample collection chamber comprises at least one volumetric marking. The at least one volumetric marking may allow a user to more accurately measure their sample as it fills the sample collection chamber. It may also mean that the device can be used to collect different volumes, depending on the purpose of the sample being provided. The at least one volumetric marking may comprise a plurality of volumetric markings. For example, the markings may include a minimum volume, a target volume, and a maximum volume markings. Such markings may allow a user to aim to provide the target volume of sample. In the exemplary case of testing for SARS-COV-2, a minimum volume marking may correspond to 0.5 ml, a target volume marking may correspond to 1 ml and a maximum volume marking may correspond to 1.5 ml.

In a set of embodiments, the device comprises a volume control arrangement which determines how far the plunger can be advanced into the sample collection chamber. This may directly control how much fluid sample can be dispensed from the sample collection chamber. The volume control arrangement may be adjustable, so that the volume dispensed from the sample collection chamber can be adjusted. The volume control arrangement may, for example, comprise an adjustable element, e.g. a nut, which engages with a portion of the sample collection conduit. The adjustable element and/or the sample collection chamber may be shaped such that the adjustable element comes into contact with the sample collection chamber, or a part within the sample collection chamber, as the sample collection conduit is advanced into the sample collection chamber. The contact may prevent further movement of the sample collection conduit and the plunger, at least in embodiments whereby the plunger is operatively coupled to the sample collection conduit. The adjustable element may be set in position during the manufacture and/or assembly of the device. The use of such a volume control arrangement may advantageously mean that the sample collection device can be used to provide a plurality of different volumes of fluid sample, thereby potentially reducing the number of different devices which need to be manufactured. In an alternative set of embodiments, the volume control arrangement may comprise a set of rings which can be attached on to the end of the sample collection conduit so as to restrict the amount by which the sample collection conduit can be advanced into the sample collection chamber, thereby restricting the amount of fluid dispensed from the sample collection chamber. The number of rings and/or the size of the rings attached to the end of the sample collection conduit may be set during or after manufacture of the device.

In embodiments wherein the inlet cap may be used to advance the plunger, as described in more detail further below, the volume control arrangement may be provided at least partially on the inlet cap. For example, the inlet cap may comprise recesses, which engage with a corresponding protrusion, e.g. locking member, on the sample collection chamber. The recesses may be located at positions on the cap which corresponding to predefined volumes being dispensed from the sample collection chamber, when the inlet cap is advanced to a position whereby the recesses are engaged by the corresponding protrusion. Accordingly, the volume of fluid dispensed may be controlled by advancing the inlet cap until the desired recess is engaged. The recesses and protrusions may form part of the restriction arrangement which is discussed further below.

The fluid sample may be dispensed from the chamber outlet into a further component, e.g. a further chamber in the form of a vial. Depending on the volume of the component into which the fluid is dispensed, and depending on the volume of fluid sample which is dispensed, sometimes there may be significant increase in pressure within the component into which the fluid is dispensed. In a set of embodiments, the sample collection device comprises a relief valve arranged to open when the plunger and/or sample collection conduit approaches a final position in the sample collection chamber. In embodiments wherein the sample collection conduit and plunger are not coupled, the relief valve may be opened when the plunger approaches a final position within the sample collection chamber. Of course, the relief valve may be opened in the final stages of movement of the sample collection conduit and/or plunger into the final position. The relief valve may be opened when the plunger and/or sample collection conduit reaches the final position in the sample collection chamber. Alternatively, the relief valve may be opened as the plunger and/or sample collection conduit approaches the final position and subsequently closed as the plunger and/or sample collection conduit reaches the final position in the sample collection chamber. Such an arrangement may advantageously allow for the release of any pressure in the final stages of movement whilst also closing of the relief valve in the final position to prevent any further fluid from leaking out of the sample collection chamber. The presence of a relief valve may be advantageous when the fluid sample is expelled out of the chamber outlet into a small vial wherein the pressure, absent the relief valve, may increase significantly. The relief valve may lower the pressure within the vial, i.e. the further component, which may reduce the pressure difference between the vial and the ambient environment. This may minimise the risk of any of the sample fluid being forcibly ejected out of the vial during separation. The relief valve may be in the form of a recess provided in a wall of the sample collection chamber and be opened to provide pressure relief when the plunger when the plunger is adjacent the recess. As the relief valve is opened, at least a portion of the fluid sample around the fluid outlet may be forced back into the sample collection chamber. This may be particularly advantageous when the fluid sample is saliva, as the saliva may otherwise collect on the chamber outlet and hang therefrom potentially causing contamination of the user of the device.

The relief valve may provide a fluid passageway between the chamber outlet and the first sub-chamber, or the second sub-chamber when the first sub-chamber has been compressed through movement of the plunger. The sample collection device may comprise further relief valves arranged to open when the sample collection conduit and/or plunger reaches the final position. A further relief valve may, for example, be positioned to provide a fluid passageway between the second sub-chamber and the third sub-chamber. The provision of additional relief valves may increase the volume into which any pressure within the further component is able to release into. The further relief valve may similarly be in the form of a recess in a wall of the sample collection chamber which is opened as the further plunger becomes adjacent the recess.

The Applicant has recognised that when providing a fluid sample, it can sometimes be difficult to determine how much sample has been provided. This is particularly the case when providing a fluid sample such as saliva which often contains air bubbles which may obscure the amount of liquid sample provided. For example, the air bubbles may produce a foam which makes it difficult to determine the liquid content within the sample collection chamber. Accordingly, in a set of embodiments, the sample collection device further comprises a float member, arranged within the sample collection chamber, configured to float on a fluid sample within the sample collection chamber. When a portion of the fluid sample comprises a liquid, the float member may have a lower density than the liquid portion of the fluid sample, but a higher density than the gaseous portion of the fluid sample. As a result, the float member will float in the liquid part of the fluid sample but sink below the gaseous, e.g. foam, part of the fluid sample. The float member may therefore be used to identify the liquid level within the sample collection chamber. The density of the float member may depend on the intended use of the sample collection device. In the exemplary case whereby the sample collection device is used to collect a saliva sample, the float member may have a density which is lower than the density of the liquid portion of the saliva, but greater than the foam portion. As a result, the float member will float in the liquid saliva within the sample collection chamber, but sit below any foam within the chamber. Liquid saliva may have a density of between 1.002-1.012 g/ml and so the float member may have a density less than 1.002 g/ml. For example, the float member may be manufactured from low density polyethylene (LDPE) which typically has a density in the range of 0.910-0.940 g/ml.

Whilst the sample collection chamber may be made from a transparent material so that a user can see into the sample collection chamber, in some instances it may nonetheless be difficult to distinguish the float member from the fluid sample. Accordingly, in a further set of embodiments, the float member has a colour distinct to the fluid sample being collected. As a result, the float member may be more identifiable within the sample collection chamber. The colour may also make the float member easier to observe through the sample collection chamber. The float member may have any colour that is distinct from the fluid sample being collected. For example, in the exemplary case of the intended use of the device being for collection of a saliva sample, the float member may be red in colour. Having a float member with a distinct colour may make it easier for a user to see the float member through the sample collection chamber, thereby allowing the user to more easily determine the fluid level within the sample collection chamber. Once a user recognises sufficient sample has been provided by observing the float member within the sample collection chamber, they may then stop providing the fluid sample. Use of a float member may therefore help to prevent overfilling of the sample collection device.

The float member may be used to indicate that sufficient fluid sample has been provided in any suitable manner. In a set of embodiments, the float member is arranged to float to a position whereby it cannot be seen when a target volume of fluid sample has been provided. Thus, a user may monitor the sample collection device, and once the float member can no longer be seen, the user may cease providing a fluid sample. In an alternative set of embodiments, the float member is arranged to float to a position whereby it can be seen when a target volume of fluid has been provided. The float member may not be visible until at least the target volume is provided. Thus, a user may monitor the sample collection device, and once the float member can be seen, the user may cease providing the fluid sample. In another set of embodiments, the sample collection chamber comprises at least one volumetric marking, and the float member is arranged to align with the volumetric markings as a fluid sample is provided into the sample collection chamber. The at least one visible marking may comprise a plurality of visible markings. This may allow the user to align the float member with different visible markings depending on the volume of fluid required.

In a set of embodiments, the float member is configured to allow fluid to pass therethrough. Allowing fluid to pass through the float member may allow the float member to float member more readily on top of the fluid sample. This may ensure that the float member accurately reflects the fluid level within the sample collection chamber as quickly as possible. The float member may be at least partially porous such that the fluid sample is able to pass through the float member. The float member may comprise at least one opening therein to allow fluid to pass through. The float member may, for example, be in the form of a ring with a hole at the centre. Such a hole may advantageously receive the sample collection conduit in embodiments wherein the sample collection conduit is arranged to move relative to the sample collection chamber. The float member may have any suitable configuration which enables it to float on top of the fluid sample. In a set of embodiments, the float member comprises a plurality of float members. Each of the float members may independently float with the fluid level and allow fluid to pass the float members and thereby fill the sample collection chamber. The provision of a plurality of float members may advantageously ensure that the float members are free to float in the fluid sample irrespective of the way in which the fluid sample is provided. A plurality of float members may reduce the change of a single float member becoming stuck in position within the sample collection chamber.

The float member may comprise a reagent stored therein. The reagent may be in solid or liquid form. The reagent may be configured such that it dissolves out of the float member and into the fluid sample when the fluid sample is provided. The reagent may interact with the sample once the fluid sample has been provided. The reagent may be released from the float member as the float member becomes soaked in the sample fluid being provided. The reagent may advantageously mix with the fluid sample. The reagent may comprise any suitable reagent. For example, the reagent may comprise a stabilization buffer arranged to stabilise the sample fluid within the sample collection chamber. The stabilization buffer may comprise a Universal Transport Medium (UTM)®. The stabilization buffer may help to ensure that the sample fluid remains stable whilst it is contained within the sample collection chamber. The reagent may comprise an agent configured to enhance the pre-analysis properties of the fluid sample. In the exemplary case of a fluid sample in the form of saliva, the reagent may comprise a lysis buffer which may separate out RNA within the fluid sample. This may reduce the amount of further processing of the sample which is required for analysis.

As discussed previously, as the fluid sample could potentially be infected, e.g. with a virus or bacteria, it may be important to avoid overfilling of the sample collection chamber so as to avoid spilling of the fluid sample onto a user's hands or surrounding work surfaces. Additionally, excess fluid within the sample collection device may prevent the proper functioning of the device. Accordingly, in a set of embodiments, at least a portion of the sample collection chamber is formed from an at least partially transparent material. The at least partially transparent material may allow a user to observe the fluid level within the sample collection chamber more easily, and the user may then stop supplying a fluid sample when sufficient sample is provided. The at least partially transparent material may be fully transparent. The portion of the sample collection chamber which is at least partially transparent may correspond to a portion of the sample collection chamber which is adjacent the float member thereby allowing the float member to be seen more easily.

In a set of embodiments, at least a portion of the sample collection chamber is defined by single skin, at least partially transparent, outer wall such that there is only one wall between the fluid sample collected within the sample collection chamber and an exterior of the sample collection device. A single-skin outer wall may allow the contents of the sample collection chamber to be seen more easily. This portion may correspond to a portion within the sample collection chamber where the float member is present. This may allow both the fluid level and the float member to be observed more easily. As a result, this may minimise the likelihood of a user overfilling the sample collection chamber, thereby minimising the risk of the fluid sample leaking out onto a user's hands or other surfaces.

In some instances, despite measures which aim to avoid the excess fluid sample being provided, it may be the case that too much fluid sample is provided via the sample collection conduit, and the fluid sample may overflow the sample collection chamber. For example, the fluid sample may overspill the first, second and third sub-chambers. Thus, in a set of embodiments, the device further comprises an overspill chamber, arranged to collect any fluid sample which overspills the sample collection chamber. The overspill chamber may extend around the sample collection chamber and be in the form of a well arranged to collect any overspill. As the fluid sample may be infected, collecting any overspill in the overspill chamber may help to avoid the spreading of any infection contained within the sample.

The opening through which the sample collection conduit extends may be partially or fully closed by the sample collection conduit itself. However, in some embodiments, the opening may be open to the external environment and may remain open during and after collection of a fluid sample. However, the Applicant has recognised that keeping the sample collection chamber open could risk the fluid sample becoming contaminated, or indeed increase the chance of the fluid sample leaking out of the sample collection chamber. Accordingly, in a set of embodiments, the sample collection device further comprises an inlet cap arranged to seal the sample collection chamber. For example, the inlet cap may close, i.e. seal, the opening of the sample collection chamber through which the sample collection conduit extends. The cap may prevent any fluid sample from escaping the sample collection chamber. In a set of embodiments, the inlet cap is pre-attached to the device before the device is used to collect a fluid sample, i.e. the inlet cap is pre-attached to the device prior so as to seal the device prior to use. Sealing the sample collection chamber in this manner may advantageously reduce the amount of packaging required as it may not be necessary to store the sample collection device in further packaging. In some embodiments, the inlet cap may be arranged to cover the sample collection conduit. This may prevent any contamination of the sample collection conduit prior to use. The inlet cap may be removed in order to provide a fluid sample. Whilst it may be preferable in some embodiments for the inlet cap to fully seal the opening of the sample collection chamber, in some instances this may not be necessary. Accordingly, in some embodiments, the inlet cap may instead close the opening of the sample collection chamber, without necessarily sealing it.

The inlet cap may seal against any appropriate part so as to seal the sample collection chamber. In a set of embodiments, the inlet cap comprises a circumferentially extending rim configured to engage with, and seal against, a corresponding circumferentially extending rim provided on a body of the device. This particular arrangement may be convenient to manufacture and achieve the desired level of sealing between the two parts. The sample collection chamber may be part of, or define, the body. In a further set of embodiments, at least a portion of the inlet cap which seals against the body of the device is made from a first material having a first hardness and the body of the device which engages with the inlet cap is made from a second material having a second hardness, wherein the first and second hardness are different. Having two materials each with a different hardness may improve the sealing as the part which is made from the harder material may cause local deformation of the part which is made from the softer material, thereby improving the seal therebetween.

In a further set of embodiments, the inlet cap is secured to the sample collection device by an inlet tamper element and wherein the inlet cap is separable from the inlet tamper element. The inlet tamper element may be in the form of a ring which extends around the sample collection chamber, and the inlet cap may be connected to the inlet tamper element by at least one breakable tab. The inlet tamper element may be used to indicate whether the inlet cap has previously been separated from the device, therefore indicating whether the device has previously been used or tampered with. For example, if the inlet cap has been separated from the inlet tamper element, this may indicate that the device has already been used and/or tampered with.

In the exemplary case of an inlet tamper element in the form of a ring, when the connection between the inlet cap and the connection ring is broken, the ring may fall away from the sample collection device. In this case, the lack of connection ring present on the device may be an immediate indicator that the device has been used or tampered with. However, in a set of embodiments, the inlet tamper element is in the form of a ring which extends around the sample collection chamber and wherein the ring is configured to remain around the sample collection chamber when the cap is separated from the connection ring. For example, the device may comprise a ring retaining feature, e.g. in the form of a protruding lug, which retains the ring around the sample collection chamber. The ring may extend around the sample collection chamber. As a result, the connection ring may act to reinforce any outer walls of the sample collection device, e.g. the sample collection chamber. Reinforcing the walls may be particularly advantageous in some embodiments wherein forces acting on the walls during operation tend to push them outwards.

In another set of embodiments, the inlet cap is configured, in at least one position, to seal the inlet to the sample collection conduit. For example, the inlet cap may seal the inlet to the sample collection conduit when it is reattached to the sample collection chamber. Sealing the sample collection conduit may advantageously form a vacuum within the sample collection conduit and thereby prevent any fluid sample from falling from within the sample collection conduit during dispensing of the sample. This may help to ensure that a fixed volume is dispensed from the device.

In another set of embodiments, when attached to the device the inlet cap is movable relative to the sample collection chamber, and wherein the inlet cap is arranged to drive the plunger into the sample collection chamber. The inlet cap may drive the plunger in any suitable manner. In embodiments wherein the plunger is operably linked to the sample collection conduit, the inlet cap may drive the sample collection conduit so as to drive the plunger. Driving the plunger with the inlet cap may beneficially mean that a user does not have to contact the sample collection conduit in order to advance the sample collection conduit into the sample collection chamber. As the sample collection conduit may be contaminated by the fluid sample, this may avoid the user contaminating their hands, for example. The cap may be coupled to drive movement of the sample collection conduit to drive movement thereof in any suitable manner. For example, the inlet cap may press against the conduit inlet in order to force the sample collection conduit into the sample collection chamber.

In a set of embodiments, the inlet cap comprises a latch arranged to latch onto the sample collection conduit when the inlet cap is advanced so as to drive the sample collection conduit into the sample collection chamber. The latch may engage a protrusion, e.g. in the form of a circumferential rim, on the sample collection conduit. The rim may be positioned at a distal end of the sample collection conduit proximal, or at, the conduit inlet. Latching of the inlet cap to the sample collection conduit may help to ensure that the inlet cap appropriately seals the sample collection conduit. The inlet cap may comprise any suitable number of latches to engage the conduit.

As discussed previously, in some embodiments the sample collection device may comprise a further plunger, the further plunger may also be advanced into the sample collection chamber by the inlet cap. Accordingly, in a set of embodiments, the sample collection device comprises a further plunger, and wherein the inlet cap is arranged to drive the further plunger into the sample collection chamber, and wherein as the further plunger is advanced into the sample collection chamber, the further plunger presses a wall of the sample collection chamber and a wall of the inlet cap together, thereby sealing the opening of the sample collection chamber. The Applicant has found that this arrangement may advantageously improve the seal between the inlet cap and the sample collection chamber. This may be achieved by having a sample collection chamber having wall which has a reduced internal diameter compared to another portion of sample collection chamber. The reduced internal diameter may be achieved by appropriately shaping the sample collection chamber and/or by having sections with different wall thicknesses. The further plunger may press the wall of the sample collection chamber against an inner wall of the cap, or an outer wall of the cap may be pressed against an inner wall of the sample collection chamber. As discussed above, the connection ring may remain around the sample collection chamber, and may act to prevent deformation of the sample collection chamber. This may further improve the seal achieved between the cap and the sample collection chamber. The further plunger may be operatively linked to the sample collection conduit and thus the further plunger may be advanced into the sample collection chamber by driving the sample collection conduit into the sample collection chamber.

In a set of embodiments, the sample collection device comprises a restriction arrangement configured to prevent movement of the inlet cap relative to the sample collection chamber in at least one direction. The restriction arrangement may advantageously control movement of the inlet cap relative to the sample collection chamber and thereby control the way in which the inlet cap is able to advance the plunger. The restriction arrangement may act to restrict movement of the inlet cap at multiple positions of the inlet cap relative to the sample collection chamber. For example, the restriction arrangement may prevent movement of the inlet cap when the inlet cap is in a first intermediate position corresponding to the sample collection conduit being in a first intermediate position and/or when the inlet cap is in a final position corresponding to the sample collection conduit being in the final position. In a further set of embodiments, the restriction arrangement is configured to prevent the inlet cap from be moved away from the sample collection chamber. Accordingly, once the inlet cap has been attached, the restriction arrangement may prevent its removal and thereby prevent further access to the fluid sample contained within the sample collection chamber. This may help to avoid tampering or contamination of the fluid sample once it has been provided.

The restriction arrangement may, for example, comprise a protrusion extending from the sample collection chamber arranged to engage with a recess provided on the inlet cap. When the protrusion is engaged within the recess on the inlet cap, movement of the inlet cap in at least one direction may be prevented. In a further set of embodiments, the restriction arrangement is configured to hold the inlet cap in at least two positions relative to the sample collection chamber. For example, the sample collection chamber may comprise a protrusion extending radially therefrom, and the inlet cap may comprise at least two recesses, axially disposed along the length of the inlet cap. As will be appreciated, the protrusion may engage with any of the recesses and thereby act to hold the inlet cap in the position corresponding to the position of the recess. The protrusion may be in the form of a circumferential protrusion, or a plurality of protrusions around the circumference of the sample collection chamber. The recess may be in the form of a circumferential recess, or a plurality of recesses around the internal circumference of the inlet cap. Of course, the protrusion may instead be provided on the inlet cap and the at least one recess may be provided on the sample collection chamber. The position of the protrusion and or recess may depend on how the inlet cap seals the sample collection chamber, i.e. whether the inlet cap extends around an outside wall of the sample collection chamber, or whether it seals against an inside wall of the sample collection chamber.

In another set of embodiments, the restriction arrangement is arranged to prevent the inlet cap from being advanced to drive movement of the plunger. Preventing the inlet cap from being advanced to move the plunger may advantageously prevent a user from inadvertently dispensing the fluid sample from the device before it is desired. The restriction arrangement may be configured to prevent advancement of the inlet cap in any suitable manner. In some embodiments, prevention of advancement of the inlet cap may be achieved by the same means that prevent retraction of the inlet cap as described above. In another set of embodiments, the restriction arrangement comprises a stop member arranged to engage with the inlet cap and prevent movement thereof, in at least one direction, and a release member coupled to the stop member and arranged to release engagement of the stop member and the inlet cap. The release member may be in the form a lever member which protrudes outwards from the device. The release member may thus be accessible to a user and may, for example, be operated by a user's finger. Whilst the stop member may prevent movement of the inlet cap, in at least one direction, the stop member may be configured such that if a sufficient force is applied to the inlet cap, the stop member may be overcome and forced out of engagement with the inlet cap such that the inlet cap can be moved relative to the sample collection chamber. This may be particularly advantageous, for example where the inlet cap is operated on by a machine, e.g. a robotic arm, which is capable of applying relatively high forces and overcoming the stop member. The stop member may stop, i.e. hold, the inlet cap in a position which corresponds to the sample collection conduit being in the first intermediate position. The stop member may engage with, e.g. abut against, an end surface of the inlet cap such that the inlet cap cannot be advanced further.

In a set of embodiments, the restriction arrangement comprises a protrusion, arranged on an inlet cap, which is arranged to abut against a body in which the sample collection chamber is provided. The body may, for example, comprise the main body of the device. The protrusion may thus prevent movement of the inlet cap relative to the sample collection chamber. The protrusion may be arranged such that the inlet cap can be moved by an initial amount, e.g. so as to engage the sample collection conduit and move the sample collection conduit partially, but positioned such that it does not cause the expulsion of sample from the sample collection chamber at the point at which the protrusion abuts against the main body. The protrusion may be in any suitable form, for example in the form of a radially extending tab or a protrusion from the surface of the inlet cap, which, due to material flexibility, may be pushed inwards to allow the inlet cap to be advanced further. In abutting against the body, the protrusion may not necessarily abut against the body directly, but may instead abut against an intermediate component arranged between the protrusion and body. Nonetheless, in abutting against the intermediate component, movement of the inlet cap will be prevented.

In order to advance the inlet cap past the position at which the protrusion abuts against the body, it may, for example, be necessary to rotate the inlet cap such that the protrusion no longer abuts against the body. In a set of embodiments, however, the protrusion can be separated from the inlet cap and/or bent relative to the inlet cap such that the inlet cap can move relative to the body. This may provide a deliberate step which a user has to perform in order to advance the inlet cap further.

The inlet cap may have any suitable form. In a set of embodiments, the body of the device is circular, the protrusion extends radially, and the protrusion extends no further than a perimeter defined by a square centred about the body, said square having sides with a length substantially matching that of the diameter of the body. Of course, the square may have sides which have a slightly larger length than that of the diameter of the body. The Applicant has recognised that by restricting the size of the protrusions in this manner may help to ensure that the sample collection device can be efficiently packaged, e.g. in packaging comprising an array of square receptacles into which the devices are arranged. Of course, while the example of a square is described, the same principle may be applied to other shapes of devices whereby the protrusion extends by a maximum extent that facilitates efficient packaging. The Applicant has recognised that it may also be beneficial in some instances to provide a cap on the chamber outlet. Accordingly, in a set of embodiments, the sample collection device comprises an outlet cap, arranged to cover the chamber outlet. The outlet cap may at least cover an end of the device in which the chamber outlet is provided. The outlet cap may act to shield the chamber outlet during use. In a set of embodiments, the outlet cap is arranged to seal an end of the device in which the chamber outlet is provided. Sealing the device in this manner may remove the need to provide additional packaging around the device. The outlet cap may be removable and thus be removed from the sample collection device when it is desired to dispense the fluid therein through the chamber outlet. The removable outlet cap may itself seal the chamber outlet, and thus removal of the outlet cap may open the chamber outlet.

However, in another set of embodiments, the chamber outlet is closed by a seal, and wherein at least part of the outlet cap is adjacent the seal such that the seal cannot be broken when the outlet cap is attached to the sample collection device. Positioning at least part of the outlet cap adjacent the seal in this manner may advantageously prevent a user from inadvertently breaking the seal and thereby prevent the expelling fluid out through the chamber outlet until the outlet cap has purposely been removed.

In a set of embodiments, the sample collection device comprises a further component connected fluidly downstream of the chamber outlet. The further component may be pre-attached to the device. The further component may be removably attached to a body of the device. In a set of embodiments, the further component is arranged to seal an end of the device in which the chamber outlet is provided. Sealing the device in this manner may mean that further packaging does not need to be provided and thus the amount of packaging required may be reduced. The further component may comprise any suitable component that may be used with the device as discussed in more detail below. For example, the further component may comprise a range of different components which it may be desirable to dispense the sample into. The further component may comprise a cap

In a further set of embodiments, an outlet tamper element is connected to a body of the device or the further component, wherein the outlet tamper element is configured such that when the further component is separated from the chamber outlet, the outlet tamper element breaks away from the body of the device or the further component. The outlet tamper element may break away entirely or partially thereby indicating that the device has been tampered with.

In a set of embodiments, the further component comprises a further chamber fluidly coupled to the chamber outlet and a protective cover surrounding the further chamber. In embodiments wherein the device comprises a connection arrangement comprising a first connection portion and a second connection portion (as described in more detail further below), the further chamber may be coupled to the first connection portion and the protective cover may be coupled to the second connection portion. The presence of a protective cover surrounding the further chamber may mean that further packaging is not required. In embodiments wherein the further component seals the end of the device, the protective cover and/or the further chamber may be the part of the further component which achieves this seal.

In a set of embodiments, the protective cover seals the end of the device at which the chamber outlet is provided, and wherein the protective cover interacts with an outlet tamper element such that if the protective cover is removed, the outlet tamper element breaks away from the body of the device or the further component. The outlet tamper element may partially or fully break away when the protective cover is removed. This arrangement may advantageously indicate tampering.

In another set of embodiments, the device comprises a removable cap attached to a distal end of the protective chamber, wherein the removable cap is configured to be attached to the end of the device when the further component is separated therefrom and/or configured to be attached to close an opening of the further chamber when it is separated from the device. The removable cap may therefore advantageously be used to close the end of the device or the further chamber. Having the cap integrally provided with the device in this manner may improve the ease of use of the device. The cap may be suitable for connection to the end of the device from which the further component is separated from, for connection to the further chamber, or suitable for connection to both.

In another set of embodiments, the device further comprises a component cap attached to a distal, closed end of the further chamber, wherein the component cap is configured for closing an open end of the further chamber. The provision of the component cap may also improve the ease of use of the device as the component cap is provided together with the device ready for use. Additionally, arranging the component cap on the end of the further chamber may mean that the component cap is aseptically stored within the protective cover, thereby ensuring that the component cap is aseptic when it is first used to cover the further chamber.

In a set of embodiments, the further chamber has an identifier arranged thereon. For example, the identifier may be a unique identifier, e.g. in the form of a QR code. The identifier may be in any suitable position on the further chamber and its position may depend on a further device which may process the further chamber and its contents. In a set of embodiments, however, the identifier is arranged on an underside of the further chamber, opposite an opening within the further chamber. Arranging the identifier on the underside of the further chamber may advantageously mean that the identifier can be read irrespective of the rotational orientation which the further chamber may be put into a further device.

In a set of embodiments, the further component comprises a fluid dispensing device. In embodiments comprising a further chamber as set out above, the further chamber may be the fluid dispensing device. The fluid dispensing device may be in the form of a dropper device which is capable of dispensing drops of liquid from the device. The provision of a fluid dispensing device may mean that the fluid sample can be accurately dispensed from the device easily. Attaching the fluid dispensing device to the chamber outlet may mean that the sample can be easily dispensed into the fluid dispensing device before being dispensed.

In another set of embodiments, the further component comprises a sample analysis device, configured to perform analysis of the sample. For example, the sample analysis device may be capable of indicating the presence of a marker, e.g. a biomarker within the sample. The sample analysis device may, for example, be capable of indicating Sars-COV-2 within the sample. Providing a sample analysis device attached directly to the chamber outlet may further improve the sample testing process as a sample may be provided by a user into the sample collection chamber, and dispensed therefrom into the sample analysis device, without any contact with the external environment. This may reduce the risk of contamination of the sample, as well as risk of contamination of the environment by the sample. The sample analysis device may be in any form, for example a vertical flow assay or a lateral flow test, or a device configured to change colour upon detecting the presence of a particular material, chemical or biological agent. In a set of embodiments, the sample analysis device indicates the presence of a biomarker using antibody-based detection techniques.

In some embodiments, the sample analysis device may comprise a point of care rapid detection (PCRD) device. In some embodiments, the sample analysis device may comprise a nucleic acid lateral flow immunoassay (NALFIA). In some embodiments, the PCRD may comprise a NALFIA.

The sample analysis device may take any suitable form, which may, for example, depend on the type of sample and analysis which is being performed. In a set of embodiments, the sample analysis device comprises a sample analysis chamber, into which the sample may be expelled, and an intermediate chamber arranged between the sample analysis chamber and the sample collection chamber outlet. The intermediate chamber may act to direct fluid into the sample analysis chamber. In a further set of embodiments, the intermediate chamber comprises a partition which forms a first intermediate chamber and a second intermediate chamber, wherein the first intermediate chamber is in fluid communication with a first portion of the sample analysis chamber and the second intermediate chamber is in fluid communication with a second portion of the sample analysis chamber. In such embodiments, the first intermediate chamber may be arranged so as to receive the fluid sample from the chamber outlet. In such embodiments, fluid may be introduced from the chamber outlet into the first intermediate chamber. The partition which forms the separate intermediate chambers which are in fluid communication with different portions of the sample analysis chamber may advantageously facilitate the outflow of air from the sample analysis chamber when the sample flows therein. The Applicant has recognised that any other suitable means for allowing the escape of air may instead be provided, for example the provision of an air valve at an end of the sample analysis chamber and/or a slot on a threaded portion of the sample analysis chamber, along which air to pass to the outside of the device.

In another set of embodiments, the sample collection device comprises a further component removably connected fluidly downstream of the chamber outlet, and an outlet tamper element connected to a body of the device or the further component, wherein the outlet tamper element is configured such that when the further component is separated from the chamber outlet, the outlet tamper element breaks away from the body of the device or the further component. In such examples, the outlet tamper element may be broken away by movement of the vial relative to the tamper element. The outlet tamper element may therefore be used to indicate whether the sample collection device has been tampered with. The further component may, for example, comprise the outlet cap described above, a vial, for example a pre-filled vial containing reagents, or any other suitable component. The further component may be pre-attached to the device.

Features of the outlet tamper element discussed below may be applied to any of the outlet tamper elements discussed above. The outlet tamper element may have any suitable form. The outlet tamper element may be in the form of a ring. In a set of embodiments, the outlet tamper element is connected to a body of the device or the further component by a plurality of breakable tabs. In a further set of embodiments, rotation of the outlet tamper element, relative to the body of the device or the further component in at least one direction is prevented by the interaction of a rotation prevention feature on the outlet tamper element and a corresponding rotation prevention feature on the body of the device or the further outlet cap. For example, the rotation prevention feature may comprise a saw-tooth shaped protrusion on the cap tamper element and the corresponding rotation prevention feature may comprise a corresponding saw-tooth shaped recess. The saw-tooth shaped protrusion and corresponding recess may prevent rotation in one direction, but permit rotation in the other direction. As such, when the further component is initially attached to the device during manufacture, e.g. by rotation, the rotation prevention features may prevent rotation of the outlet tamper element, thereby avoiding breaking the breakable tabs. Conversely, when the further component is removed, the rotation prevention features may allow rotation in the direction in which the outlet cap is undone, and thereby the breakable tabs may break, thus indicating that the outlet cap has been removed and that the device has been used and/or tampered with.

The fluid sample may be dispensed directly from the sample collection device into another chamber in which further processing or analysis of the sample may be performed. Depending on the particular application, it may be possible to simply hold the sample collection device, specifically the chamber outlet, above a further device into which the sample is to be dispensed. However, this may not be appropriate in some instances, particularly where the sample may be infected, e.g. with a virus or bacteria, and so it may be desirable to contain the fluid sample as far as possible. Accordingly, in a set of embodiments, the sample collection device further comprises a connection arrangement arranged fluidly downstream of the chamber outlet, for connecting a further component to the sample collection device. As such, a further component may be attached to the device. The outlet cap described above may be attached to the connection arrangement. The further component may, for example, comprise a container, e.g. a vial, into which the fluid sample may be dispensed, or any other component whereby it may be desired to transfer fluid. For example, the further component may comprise a fluid connector attached to a fluid hose. The further component may, for example, comprise a sample analysis chamber which may, for example, comprise a reagent therein.

It may be desirable in some instances to be able to attach different types of further component, each having a different connection portion, to the connection arrangement. Accordingly, in a set of embodiments, the connection arrangement comprises a first connection portion, for connecting a first further component having a complementary first connection portion, and a second connection portion for connecting a second further component having a complementary second connection portion. Therefore, as will be appreciated by those skilled in the art, further components having two different connection portions may be connected to the connection arrangement. This may increase the application of the sample collection device, potentially meaning that the sample collection device is compatible with an increased number of further components. The first connection portion and second connection portion may have any suitable format. For example, the first and second connection portion may be configured to achieve a friction fit with the corresponding first and second components. In this case, the first connection portion may have a smaller dimension than the second connection portion. In a set of embodiments, the first connection portion comprises a thread having a first diameter, e.g. 12 mm, and the second connection portion comprises a thread having a second, larger, diameter, e.g. 16 mm. As such, components with different sized external threads may be connected to the connection arrangement. This may allow a single sample collection device to be used with a wider variety of different components. Of course, the connection arrangement may comprise further connection portions.

In a set of embodiments, the chamber outlet comprises a seal which prevents the passage of fluid therethrough. The seal may therefore prevent the passage of fluid until it is desired to expel the fluid from the device. The seal may be overcome or removed in any suitable manner in order to allow fluid to pass out of the chamber outlet. For example, the seal may be manually removed by a user pulling or tearing the seal away from the sample chamber outlet. However, in a set of embodiments, the device comprises a seal arranged to seal the sample chamber outlet, the sample collection conduit is movable relative to the sample collection chamber, and wherein the sample collection conduit is configured to break the seal. Breaking the seal with the sample collection conduit may provide a convenient means for overcoming the seal and avoid a user having to come into contact with the sample chamber outlet which could potentially cause contamination of the sample. As movement of the sample collection conduit may also be used to advance the plunger, using the sample collection conduit to break the seal may advantageously break the seal at the appropriate time in order to allow the fluid to be expelled from the sample collection chamber. The sample collection conduit may be configured to break the seal in any appropriate manner. For example, the sample collection conduit may comprise a pointed tip configured to break, e.g. penetrate, the seal.

The sample collection conduit may comprise a mouthpiece configured to allow a user to seal their mouth around the sample collection conduit when providing an oral sample. Such a mouthpiece may allow a user to achieve a better seal around the sample collection conduit when providing a sample and thus the chance of leaking of the sample may be reduced. As the sample may comprise infected material, reducing the risk of any leakage, which could potentially infect others, is particularly advantageous.

The sample collection device may have various different structural forms. In a set of embodiments, a wall of the device comprises a deformation feature which allows deformation of the wall during manufacture of the device. Facilitating deformation may allow a tool, e.g, a mould, to be removed more easily from the device without risking damage to the device itself. In a further set of embodiments, the device comprises an overspill chamber which extends at least partially around the sample collection chamber, and wherein the wall comprising the deformation feature is a wall of the overspill chamber. In a further set of embodiments, the wall comprises a protrusion and wherein the deformation feature is arranged so as to facilitate deformation of the wall around the protrusion. The Applicant has recognised that the deformation feature may facilitate the presence of a protrusion in a position within the device which may otherwise not be possible due to manufacturing constraints.

Whilst the deformation feature may allow structures within the device which may have otherwise not been possible, the deformation feature may weaken the device. Accordingly, in a set of embodiments, the device further comprises a reinforcing element which extends around the wall and reinforces the deformation feature. The reinforcing element may be attached to the device at a stage during manufacture after which a tool, e.g. a mould, has been removed from the device. The reinforcing element may act to reinforce the wall and prevent, or at least reduce the amount of, deformation of the wall due to the deformation feature. In a set of embodiments, the reinforcing element comprises a label which extends around the wall. The label may, for example, include operation instructions arranged thereon. The label may thus function both to reinforce the wall of the device, and also provide instructions for a user. The label may be attached using any suitable means, e.g. an adhesive.

As discussed above, further components may be attached to the device which comprise a reagent arranged therein. In addition, or alternatively, in a set of embodiments, the sample collection chamber comprises a reagent arranged therein. The reagent may be any reagent which may be used in as part of the analysis or pre-analysis process of the sample. The reagent may, for example comprise a buffer solution, e.g. a UTM®. Arranging the reagent within the sample collection chamber may allow at least part of the analysis or pre-analysis process to occur before the sample is even expelled out of the sample collection chamber. In some embodiments, the reagent may comprise: a UTM, synthesis primers with desired labels for amplification of nucleic acids, nano-particles configured to bind to specific targets on nucleic acids and/or any other suitable chemical agent aimed at priming or altering the sample, e.g. a saliva sample, as part of the analysis process.

The reagent may be freely contained within the sample collection chamber such that it interacts with the sample as soon as the sample enters the sample collection chamber. However, for some reagents, this may not be desirable. Accordingly, in a set of embodiments, the reagent is arranged within a sealed container which can be selectively opened by a user. Arranging the reagent in this manner may advantageously provide the ability for a user to determine when they wish for the sample to mix with the reagent. Once the sealed container has been opened, the device may be shaken in order to mix the sample with the reagent. The sealed container may comprise any suitable container, e.g. a capsule. In a set of embodiments, the container is in the form of a blow-fill-seal container. Such a container may be relatively inexpensive to manufacture. For reagents which are temperature sensitive, a container with a seal arranged therein may be used. The sample collection conduit may be configured to penetrate said seal in order to facilitate mixing of the sample and the reagent. Whilst one sealed container is described, it will be appreciated that the device may comprise a plurality of sealed containers arranged within the sample collection chamber. Each sealed container may, for example, house a different reagent.

Opening of the sealed container to allow mixing may be achieved in any suitable manner. In a set of embodiments, the sample collection conduit is arranged to open the sealed container when the sample collection conduit is advanced into the sample collection chamber. The sample collection conduit may, for example be configured to penetrate, and thereby open, the sealed container. This arrangement may provide a convenient means for breaking the sealed container. The device may be configured such that it is possible to open the sealed container without expelling the sample from the sample collection device. This may, for example, comprise opening the sealed container before opening/penetrating a chamber seal provided on the chamber outlet. In such embodiments, the sample collection conduit may penetrate the sealed container before penetrating the chamber seal. In embodiments comprising a restriction member which restricts movement of the inlet cap, the restriction member may restrict movement of the inlet cap, and hence the sample collection conduit, such that the inlet cap is held at a position wherein the sample collection conduit has penetrated the sealed container, but not penetrated the chamber seal. The restriction member may then be released in order to advance the inlet cap, and hence the sample collection conduit, past this position.

In embodiments which comprise a plurality of sealed containers each housing a reagent, the sealed containers may be arranged such that they are opened/penetrated in a specific order. The sample collection conduit may, for example, be arranged to open/penetrate the sealed containers in the specific order such that the reagents are released in a desired order. For example, the sealed containers may effectively be stacked on top of one another such that the containers are opened/penetrated in sequence. In some embodiments, the inlet cap may comprise a restriction arrangement, e.g. multiple restriction members, which is capable of controlling the advancement of the sample collection conduit such that each sealed container can only be opened upon operation of the appropriate part of the restriction arrangement, e.g. by releasing the appropriate restriction member.

Whilst a reagent is described, it will be appreciated in other embodiments, any other medium which may interact with a sample may instead be arranged within the sample collection chamber and/or the sealed container.

The sample collection conduit may comprise a tapered friction fitting at its inlet and/or the chamber outlet may comprise a tapered friction fitting. The tapered fitting may, for example, conform to the Luer or ENFit standard. This may advantageously allow a sample to be inserted into the sample collection device, through the sample collection conduit, if the sample is contained within other means having a fitting capable of connecting with the tapered friction fitting. Similarly, the fluid may dispensed into other means having a fitting capable of connecting with the tapered friction fitting on the chamber outlet. The sample collection conduit or chamber outlet may comprise other connection means, e.g. such as a threaded portion. Such a threaded portion may conform to the Luer-lock or ENFit standard.

The sample collection device may further comprise an adaptor configured to be attached fluidly downstream of the chamber outlet, e.g. on the connection arrangement. The adaptor may allow the sample collection device to be used to transfer the sample to any one of a number of different components or devices. This may advantageously increase the number applications the sample collection device can be used with.

The sample collection device may be made from any suitable material or combination of materials. As mentioned above, part or all of the sample collection device may be at least partially transparent, e.g. fully transparent, such that the user can see their sample as it passes into the device. The sample collection chamber may be made from polypropylene and the plunger may be made from polyethylene or polycarbonate. At least in those embodiments wherein the plunger(s) and conduit are integrally formed, the conduit may also be made from polyethylene or polycarbonate. Such an embodiment may advantageously remove the need to provide an O-ring between the plunger(s) and the sample collection chamber, as well as removing the need to provide a lubricant to lubricate the movement of the plunger(s). Removing such lubricant may be advantageous as the lubricant may potentially affect the results of any testing performed on the sample.

Whilst the various parts of the device have been described as being attached together above, the device may be provided with each of the component separately, e.g. as a kit of parts. For example, the kit of parts may comprise the sample collection chamber with the sample collection conduit and plunger(s) inserted therein, provided with a separate sample analysis chamber which may be selectively attached by the user as desired. Any combination of different components as described above may be provided in the kit of parts.

As will be appreciated by those skilled in the art, whilst the device may be particularly well suited to the collection of an oral fluid sample, as described in some of the embodiments above, the device may also be used for the collection, measurement and transfer of other fluid samples, other than oral fluid samples. For example, fluid samples may be transferred directly into the device, e.g. from another device such as a syringe.

According to another aspect of the present invention there is provided a medical device comprising: a chamber for receiving a fluid, wherein the chamber comprises a first opening at one end of the chamber and a second opening at a second end of the chamber; a first removable cap pre-attached to the chamber to close the first opening; and a second removable cap pre-attached to the chamber to close the second opening. Having a chamber which is closed by pre-attached caps may seal the inside of the chamber. This may advantageously mean that secondary packaging may not be required to store the device as the chamber may remain aseptic until one of the caps is removed. The first and second caps may seal the first and second openings, respectively. Each of the first and second caps may interact with a respective tamper element which indicates whether the cap has been removed from the chamber. The tamper element may have any of the features of the tamper elements described above. Use of a tamper element may advantageously indicate to a user whether the device has been tampered with. Each cap may also comprise any of the features of the inlet cap described in the embodiments above.

Some preferred embodiments of the present invention will now be described, by way of example only, and with reference to the following drawings, in which:

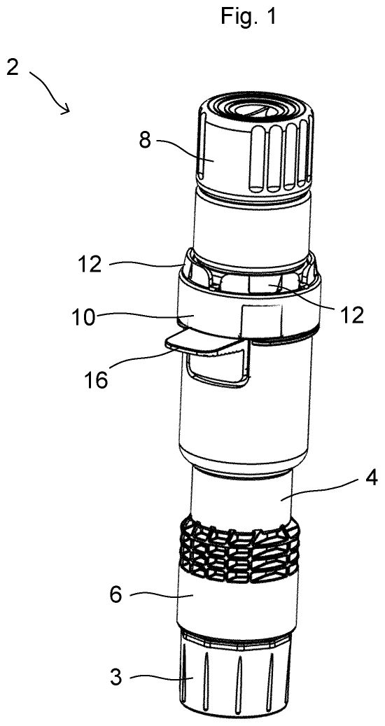

FIG. 1 shows a perspective view of a sample collection device in accordance with an embodiment of the present invention;

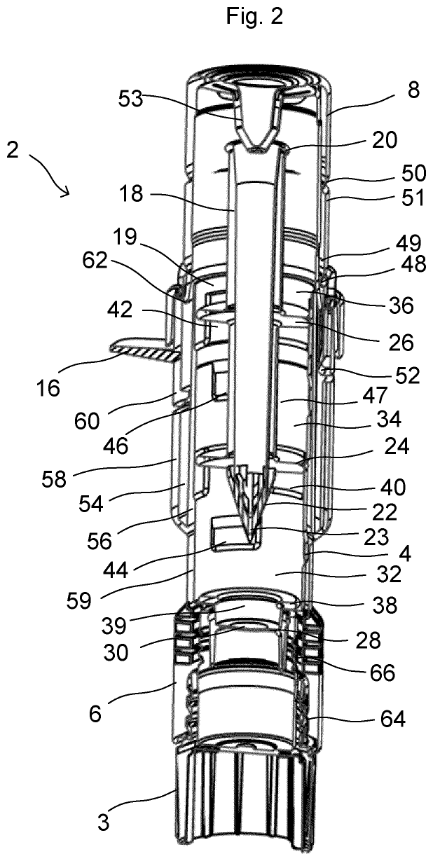

FIG. 2 shows a cross-sectional view through the sample collection device shown in FIG. 1;

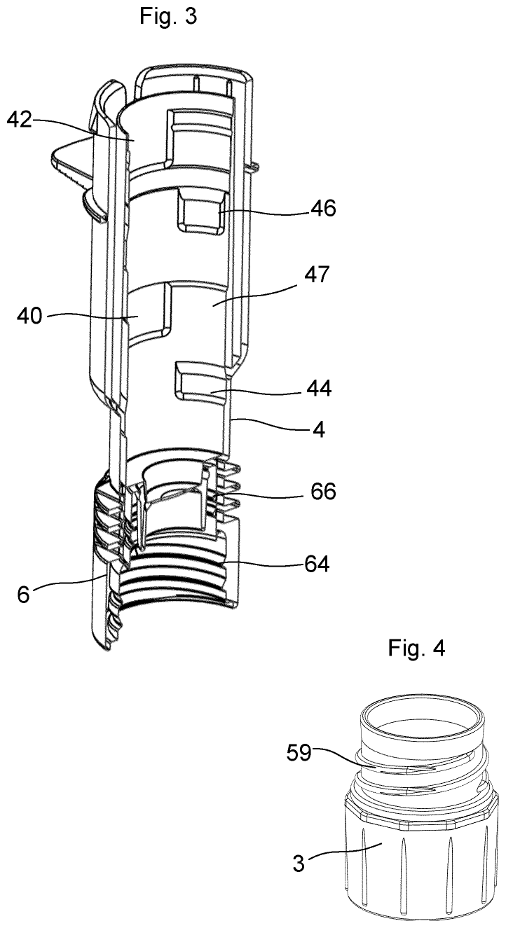

FIG. 3 shows a cut-away view of the sample collection chamber of the sample collection device shown in FIG. 1;

FIG. 4 shows a perspective view of the chamber outlet cap shown in FIG. 1;

FIG. 5 shows a perspective view of the sample collection device shown in FIG. 1 focussing on an upper portion thereof;

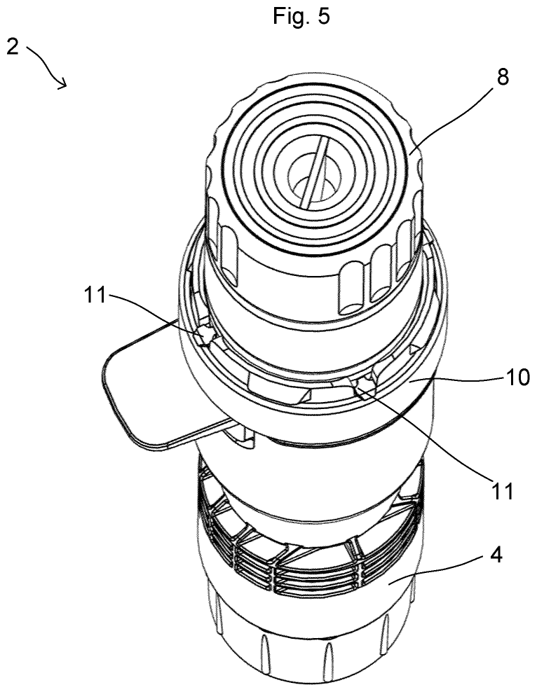

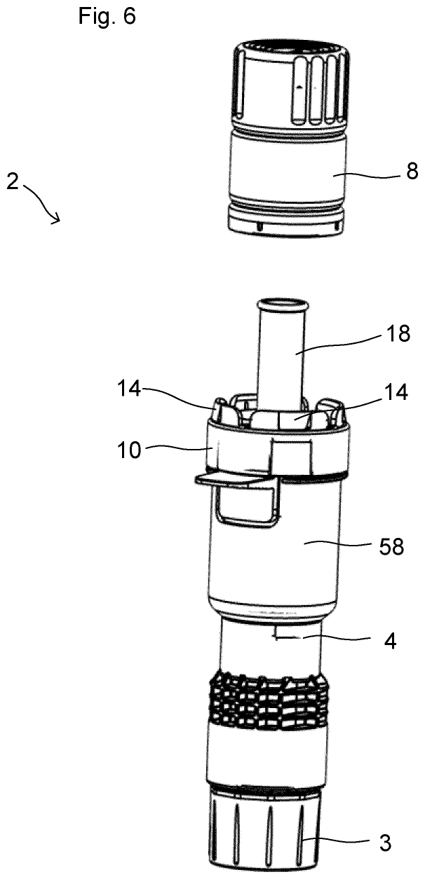

FIG. 6 shows a perspective view of the sample collection device shown in FIG. 1 with the cap removed;

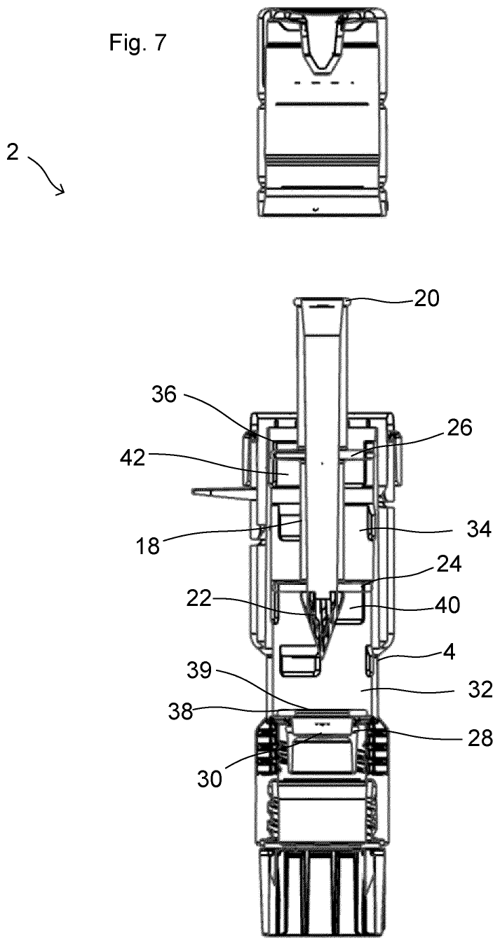

FIG. 7 shows a cross-sectional view through the sample collection device shown in FIG. 1 with the cap removed;

FIG. 8 shows a cross-sectional view through the sample collection device illustrating how the float member floats as the fluid level increases;

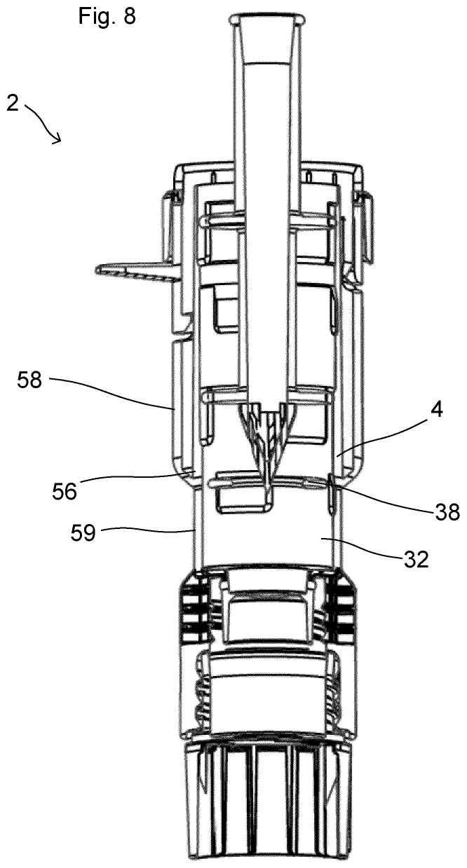

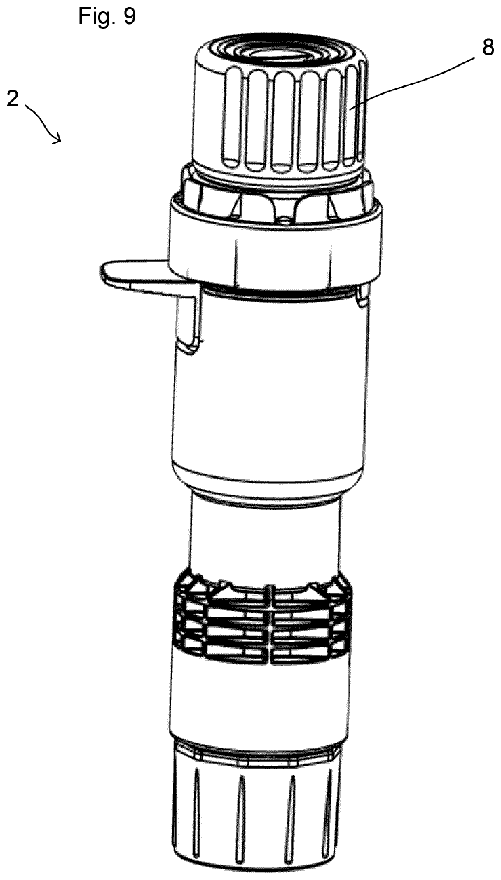

FIG. 9 is a perspective view of the sample collection device shown in FIG. 1 with the cap advanced into a first intermediate position;

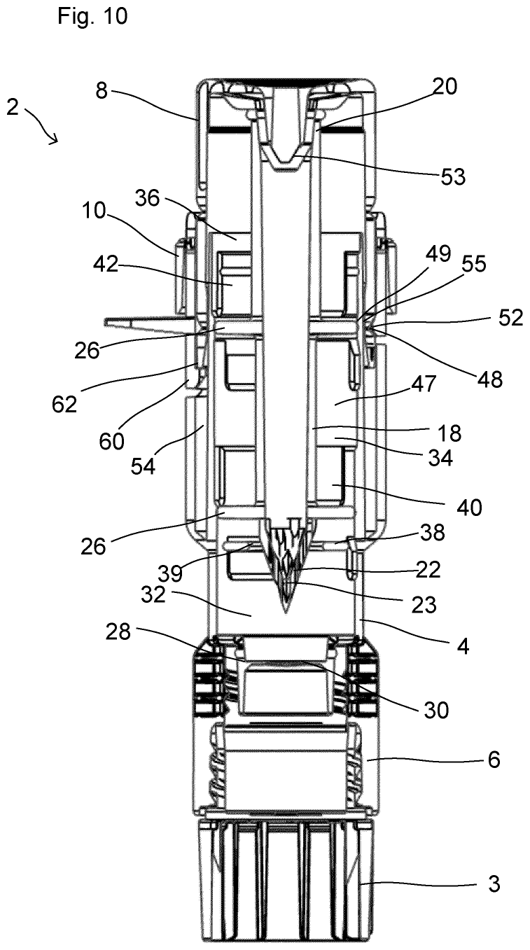

FIG. 10 is a cross-sectional view through the sample collection device with the cap in the position shown in FIG. 9;

FIG. 11 is a perspective view of the sample collection device shown in FIG. 1 with a sample analysis chamber attached thereto;

FIG. 12 is a cross-sectional view through the sample collection device and sample analysis chamber shown in FIG. 11;

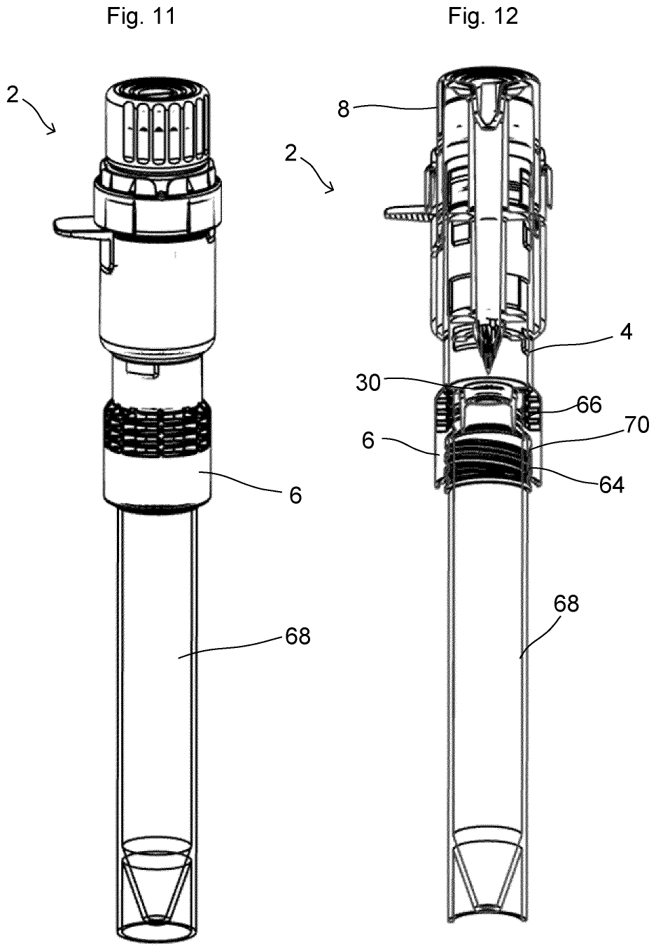

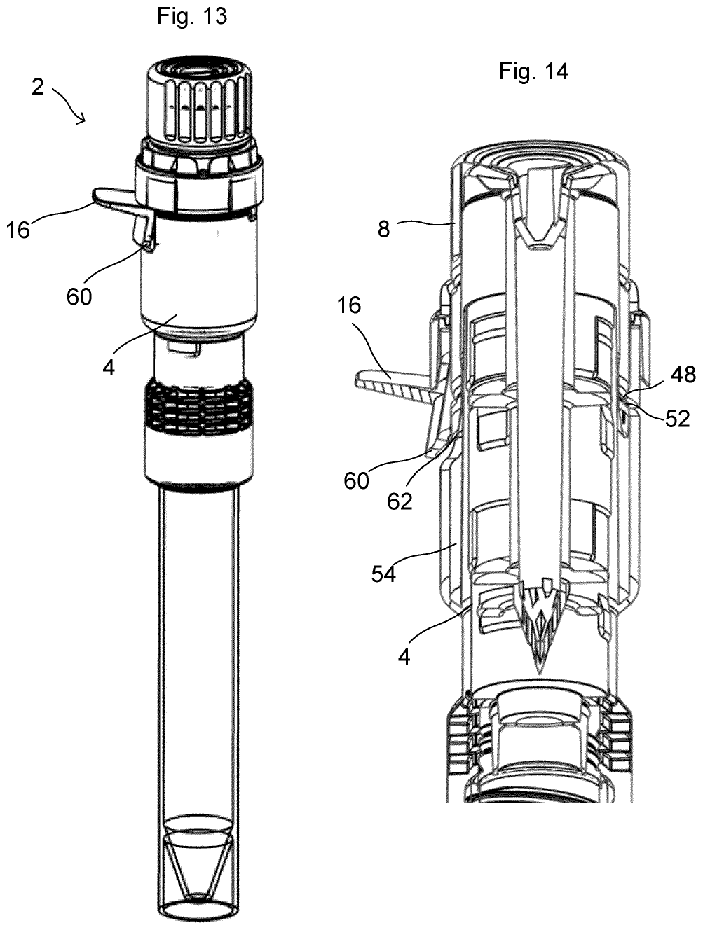

FIG. 13 is a perspective view of the sample collection device with a release member moved into a released position;

FIG. 14 is a cross-sectional view of the sample collection device with the release member in the released position;

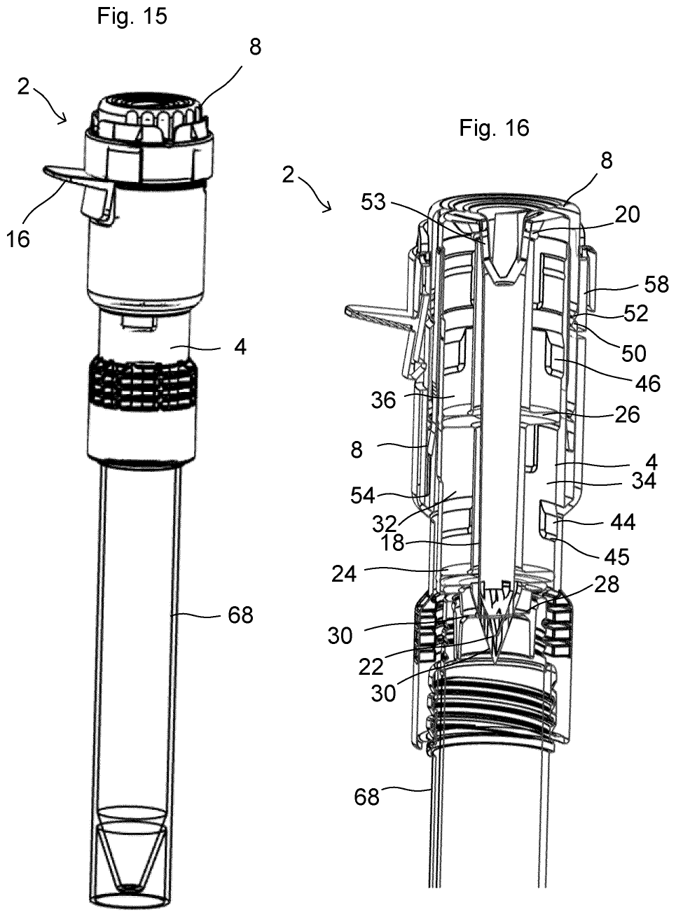

FIG. 15 is a perspective view of the sample collection device with the sample collection conduit in the final position;

FIG. 16 is a cross-sectional view of the sample collection device with the sample collection conduit in the final position;

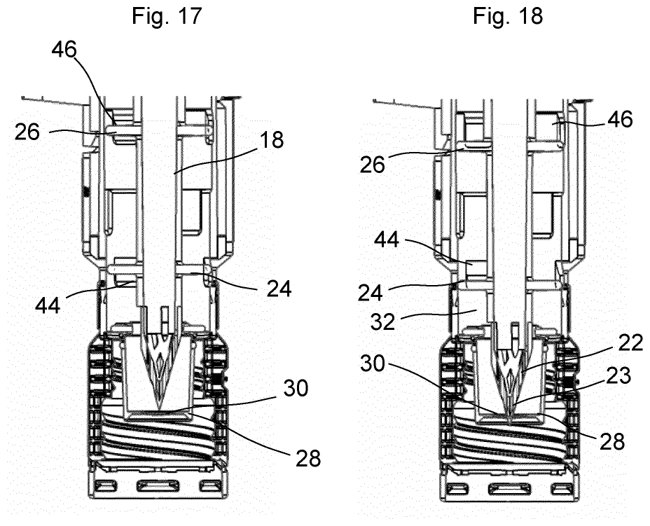

FIG. 17 shows a cross-sectional view of the sample collection device of FIG. 1 wherein the third and fourth fluid passageways are open;

FIG. 18 shows a cross-sectional view of the sample collection device of FIG. 1 wherein the third and fourth fluid passageways are closed;

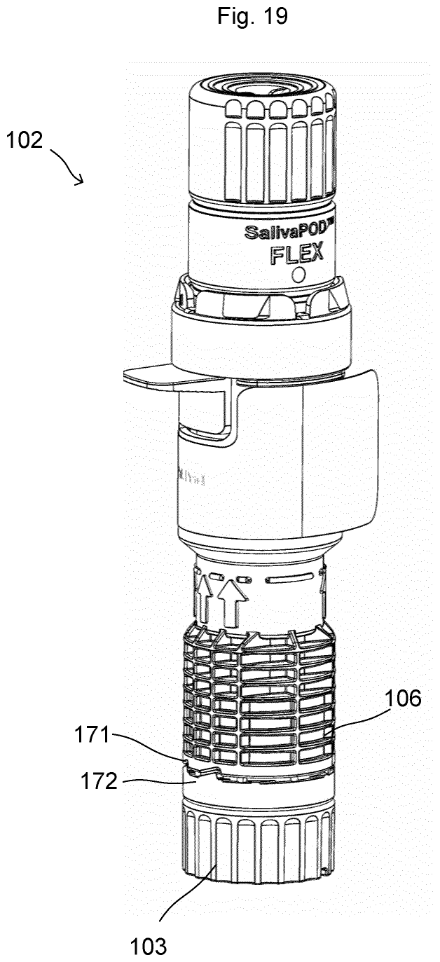

FIG. 19 shows a perspective view of a sample collection device in accordance with another embodiment of the present invention;

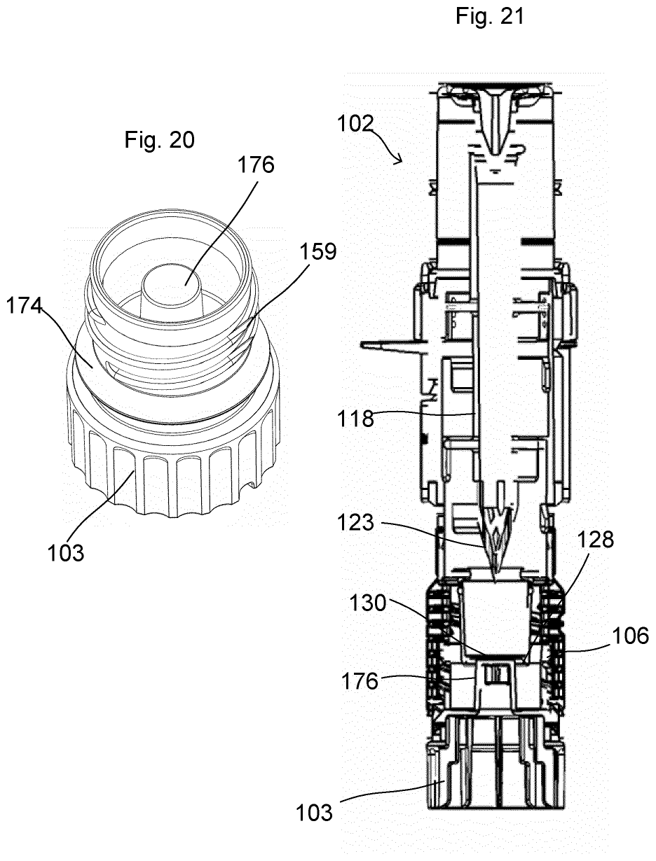

FIG. 20 shows a perspective view of the outlet cap on the sample collection device shown in FIG. 19;

FIG. 21 shows a cross-sectional view of the sample collection device shown in FIG. 19;

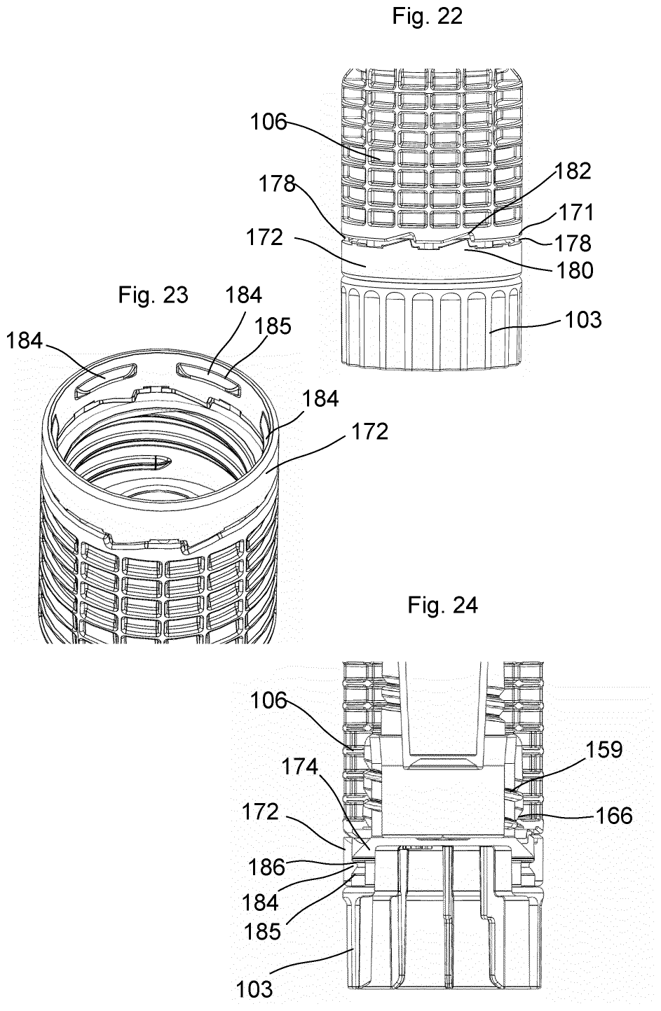

FIG. 22 is a view focussing on the outlet cap and its connection to the connection arrangement;

FIG. 23 is a perspective view of the connection portion with the connection arrangement with the outlet cap removed;

FIG. 24 is a cross-sectional view focussing on the connection arrangement and outlet cap;

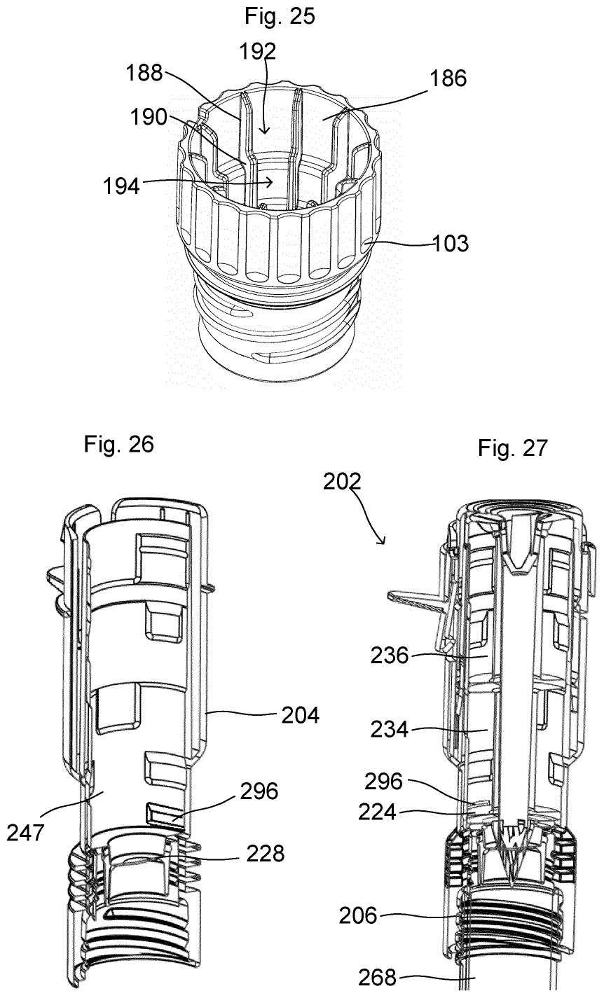

FIG. 25 is an underside view of the outlet cap, of the embodiment depicted in FIG. 19, in isolation;

FIG. 26 is a cut-away view of a sample collection chamber in accordance with another embodiment of the present invention which comprises a relief valve;

FIG. 27 is a cut-away view of the sample collection chamber of FIG. 26 with a plunger fully advanced therein;

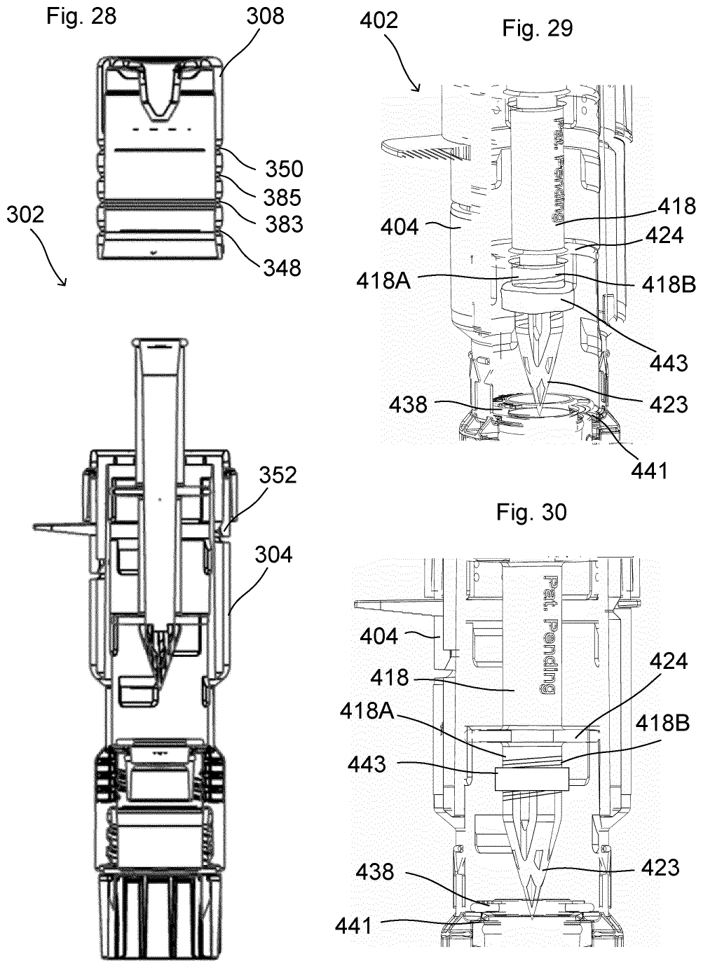

FIG. 28 is a cross-sectional view of a sample collection device comprising a volume control arrangement in accordance with another embodiment of the present invention;

FIG. 29 is a cut-away view of a sample collection device comprising an alternative volume control arrangement, in accordance with another embodiment of the present invention;

FIG. 30 is a cross-sectional view of the sample collection device shown in FIG. 29

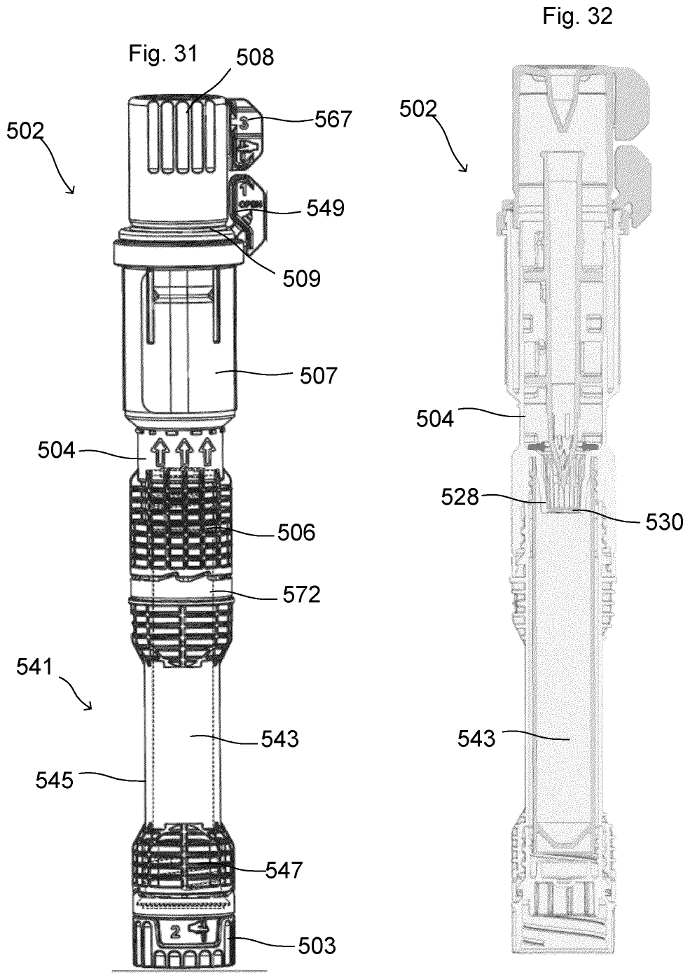

FIG. 31 is a perspective view of a sample collection device in accordance with an embodiment of the present invention;

FIG. 32 is a cross-sectional view of the sample collection device shown in FIG. 31;

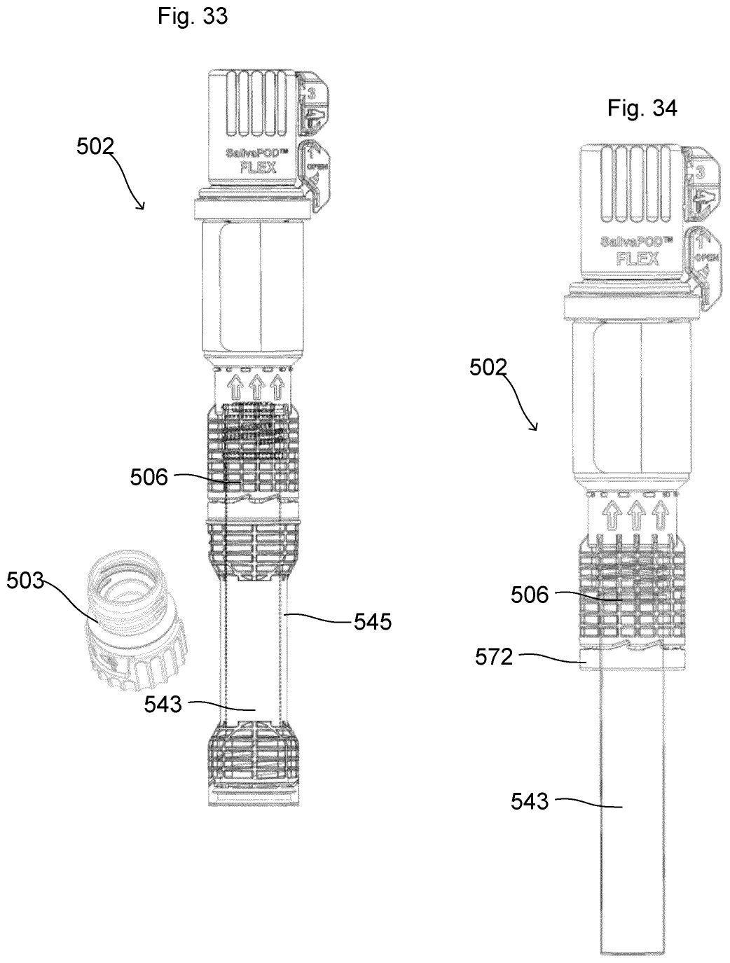

FIG. 33 is a perspective view of the sample collection device of FIG. 31 with the lowermost cap removed;

FIG. 34 is a perspective view of the sample collection device of FIG. 31 with the protective cover removed therefrom;

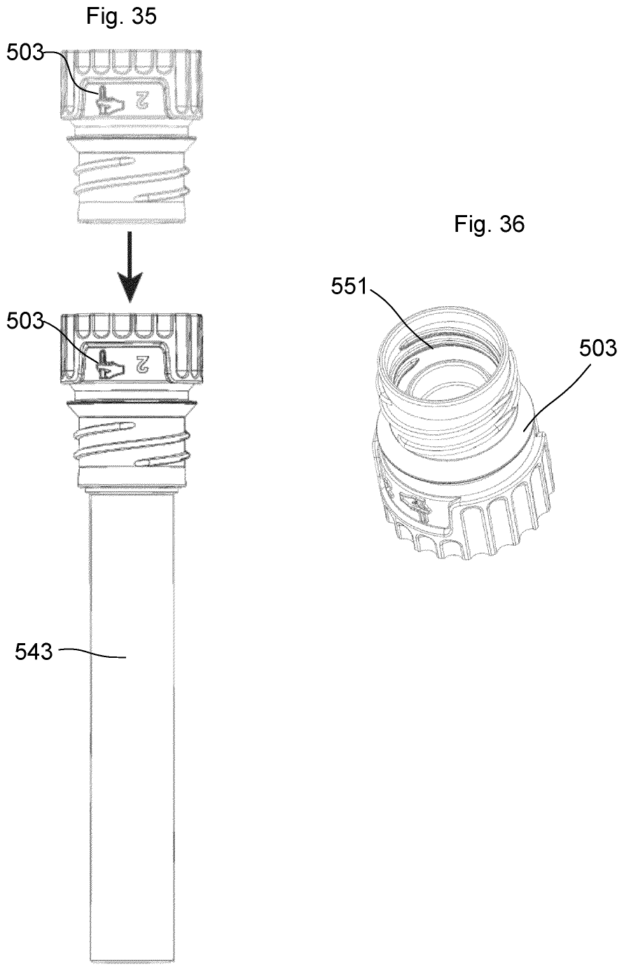

FIG. 35 illustrates a further chamber with a cap being connected thereto;

FIG. 36 is a perspective view of the cap shown in FIGS. 31-35

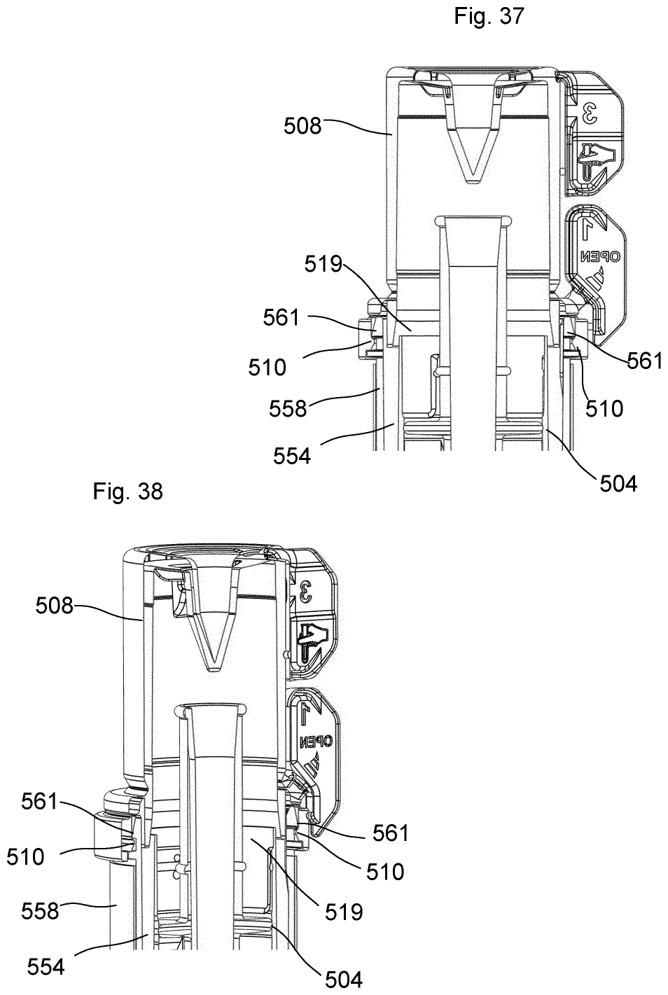

FIG. 37 and FIG. 38 are cross-sectional views through the uppermost part of the sample collection device shown in FIG. 31;

FIG. 39 is a perspective view of the sample collection device shown in FIG. 31 with the label removed to reveal the deformation feature;

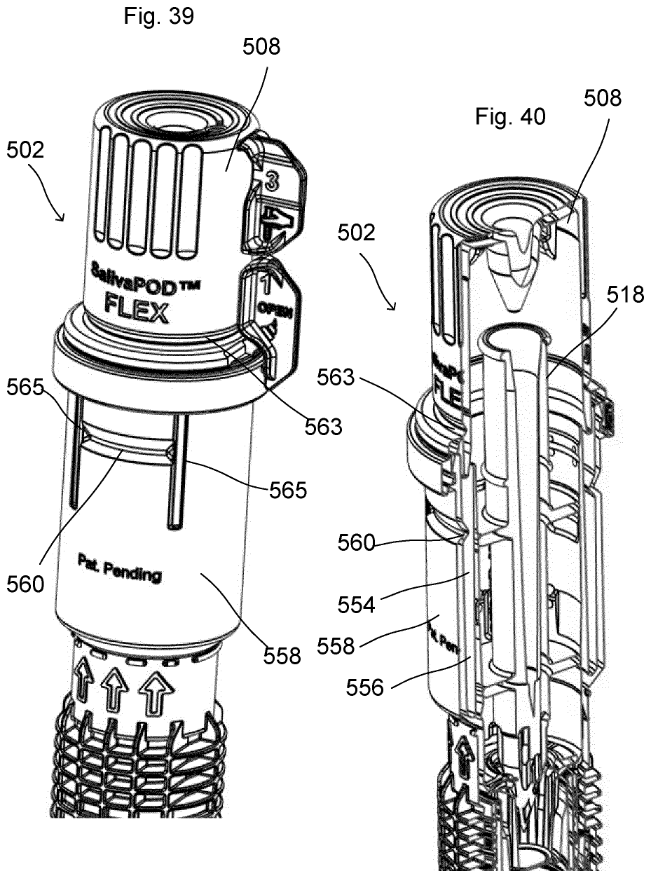

FIG. 40 is a cross-sectional view through the sample collection device shown in FIG. 31;

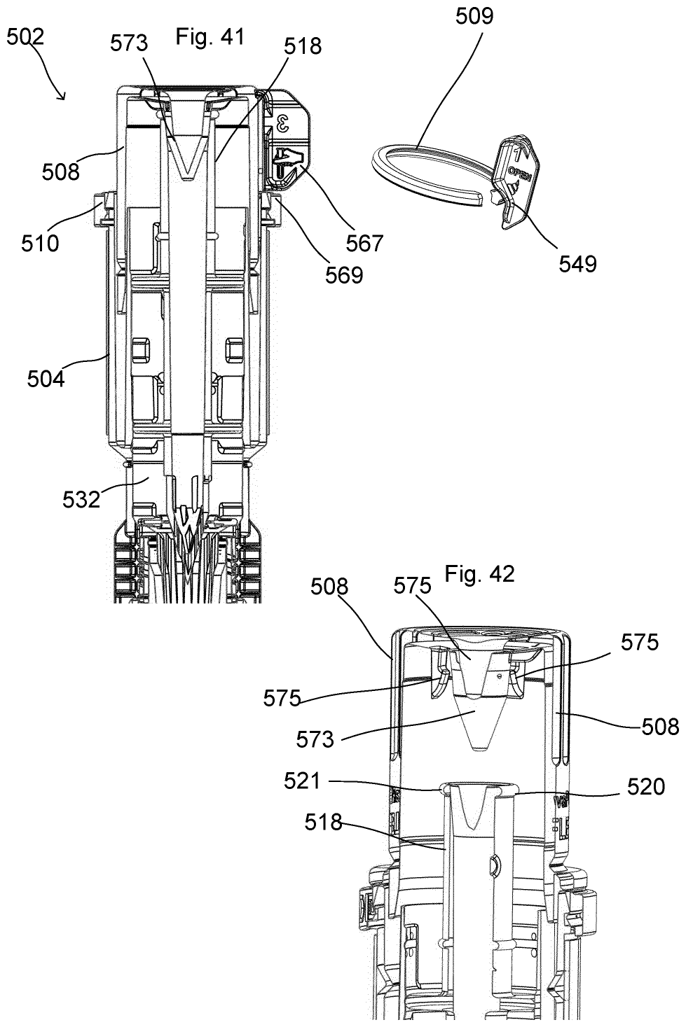

FIG. 41 is a cross-sectional view through the upper part of the sample collection device shown in FIG. 31 with the cap in an advanced position;

FIG. 42 is a cross-sectional view through the sample collection device shown in FIG. 31 focussing on the latch members;

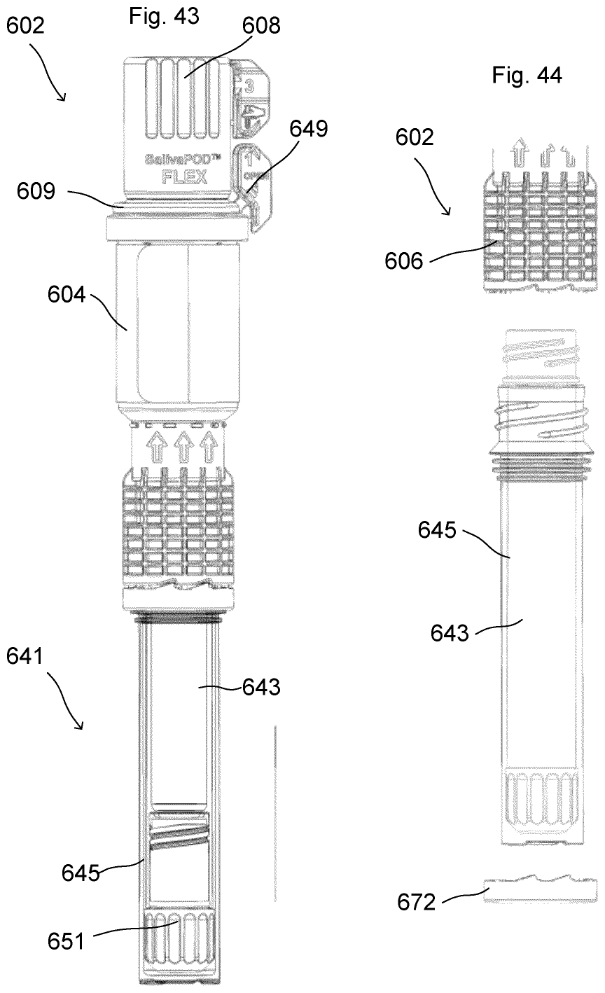

FIG. 43 is a side-on view of another embodiment of a sample collection device before use;

FIG. 44 is a side-on view of the sample collection device in FIG. 43 after a sample has been collected and it has been ejected into the further component;

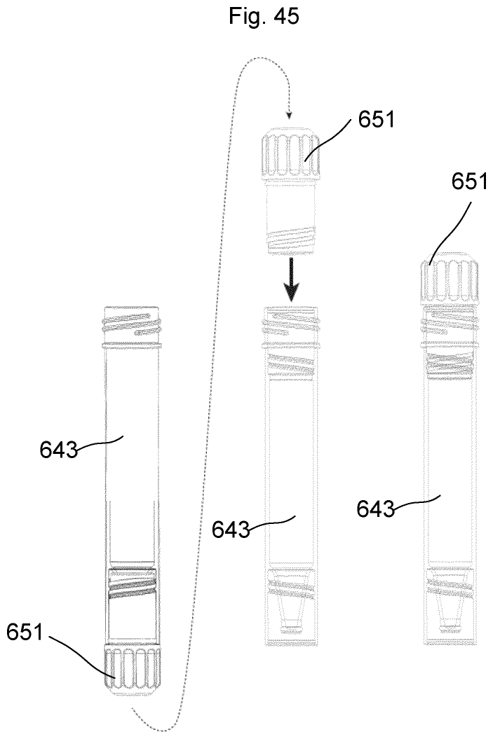

FIG. 45 is a perspective view of the further component containing a sample and with the lid thereon;

FIG. 46A is a view of another embodiment of a sample collection device comprising a further component but without any cover thereon, FIG. 46B is a view of the further component separated from the device and closed with a cap;

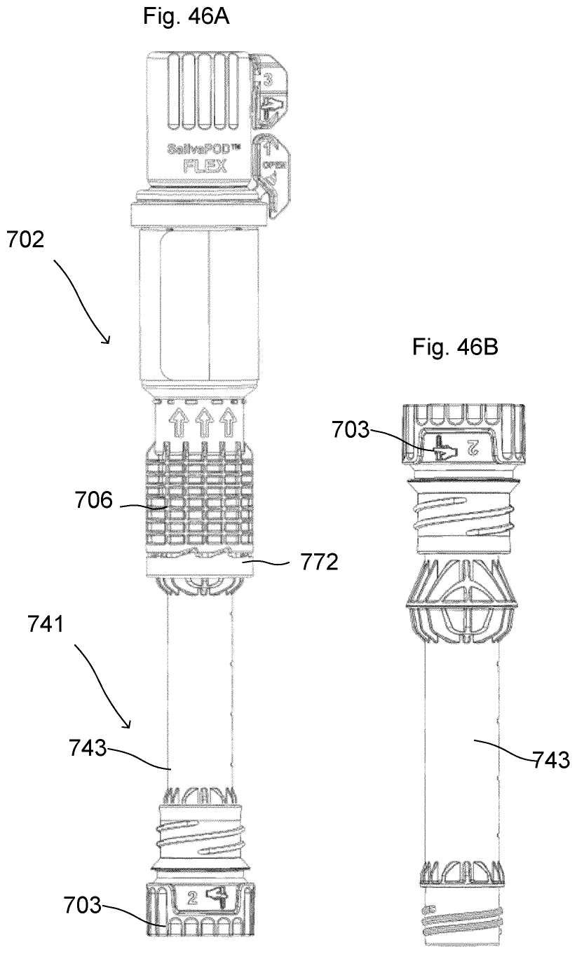

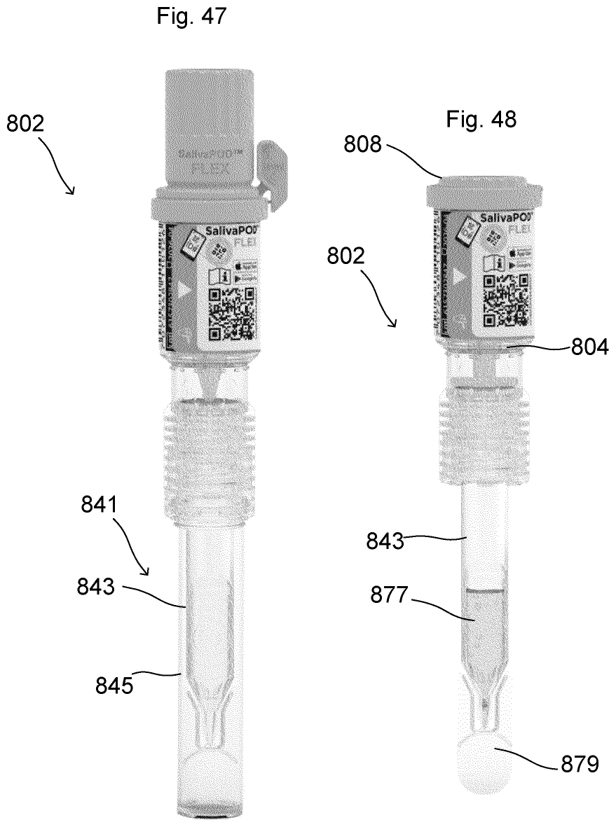

FIG. 47 is a view of another embodiment of a sample collection device in which the further component attached to the sample collection chamber is in the form of a dropper;

FIG. 48 is a side-on view of the sample collection device shown in FIG. 47 with a sample contained therein and with an outer cover removed;

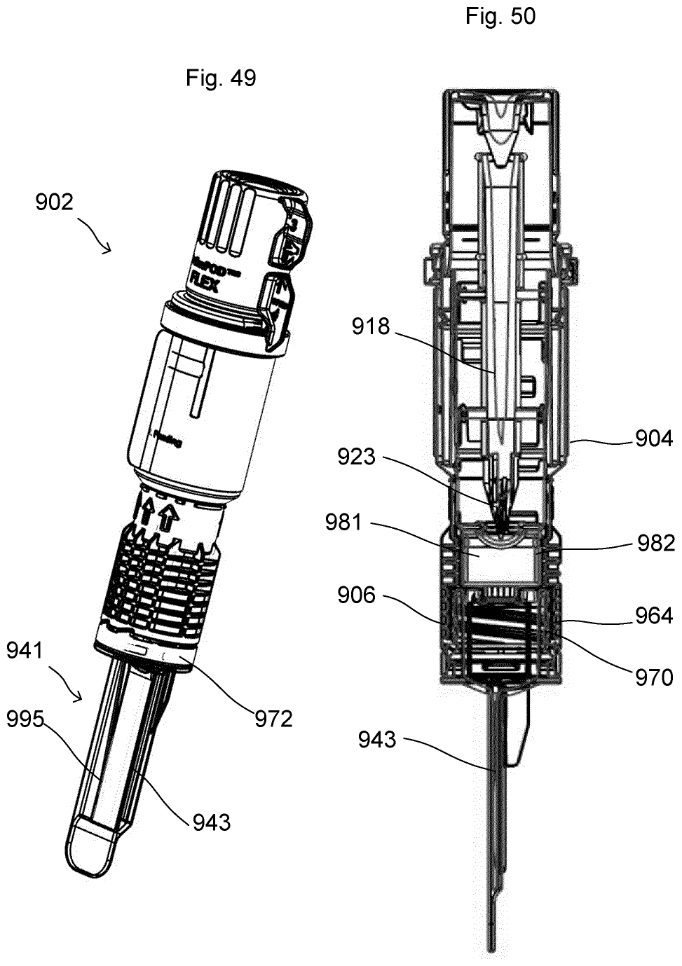

FIG. 49 is a perspective view of another embodiment of a sample collection device comprising a further component in the form of a testing chamber attached to the sample collection chamber;

FIG. 50 is a cross-sectional view through the sample collection device shown in FIG. 49;

FIG. 51 is a cross-sectional view of the testing chamber separated from the other components of the device;

FIG. 52 is a cross-sectional view of the testing chamber;

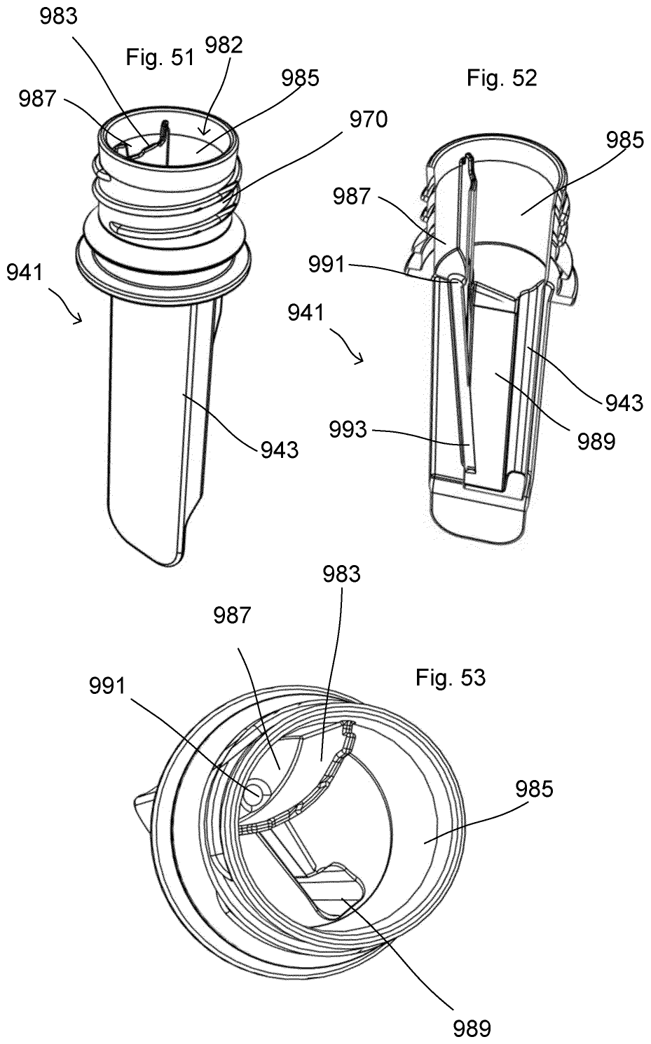

FIG. 53 is a view looking into the testing chamber at an angle and from above;

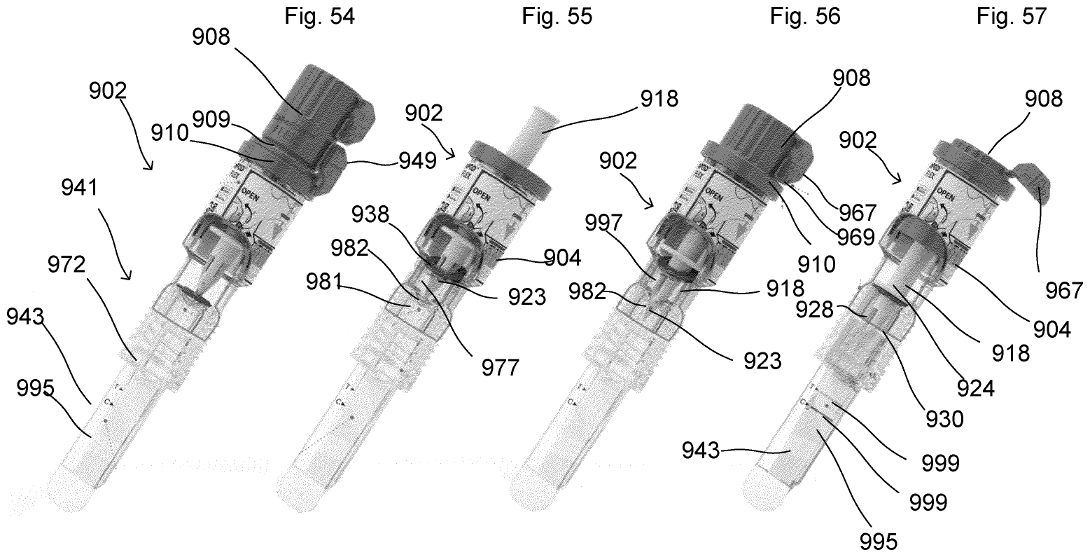

FIGS. 54-57 show operation of the sample collection device shown in FIG. 49;

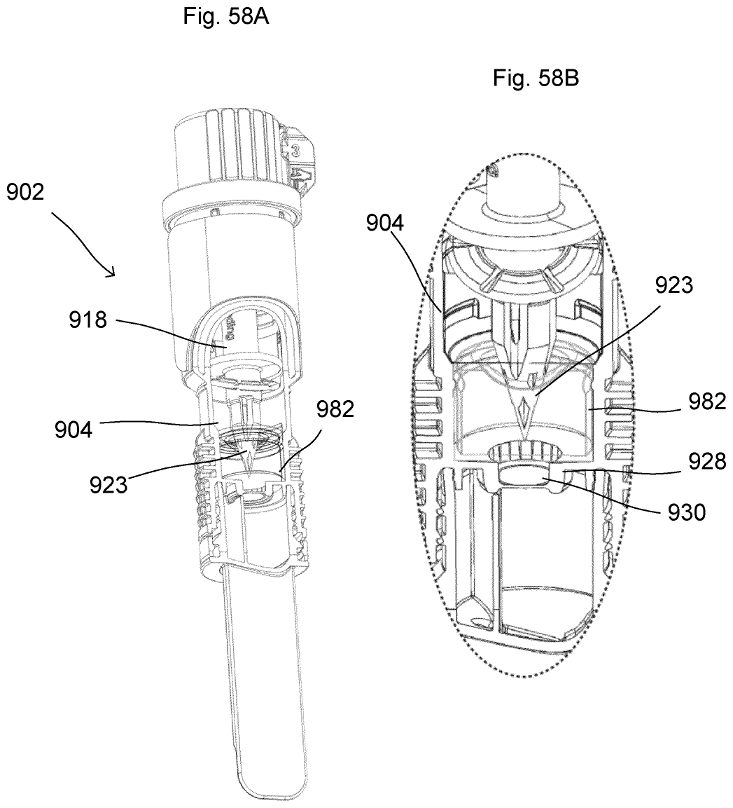

FIG. 58A shows a partial cut-away view through the sample collection device with the sealed container penetrated; FIG. 58B shows a detailed view focussing on the sealed container inside the sample collection chamber;

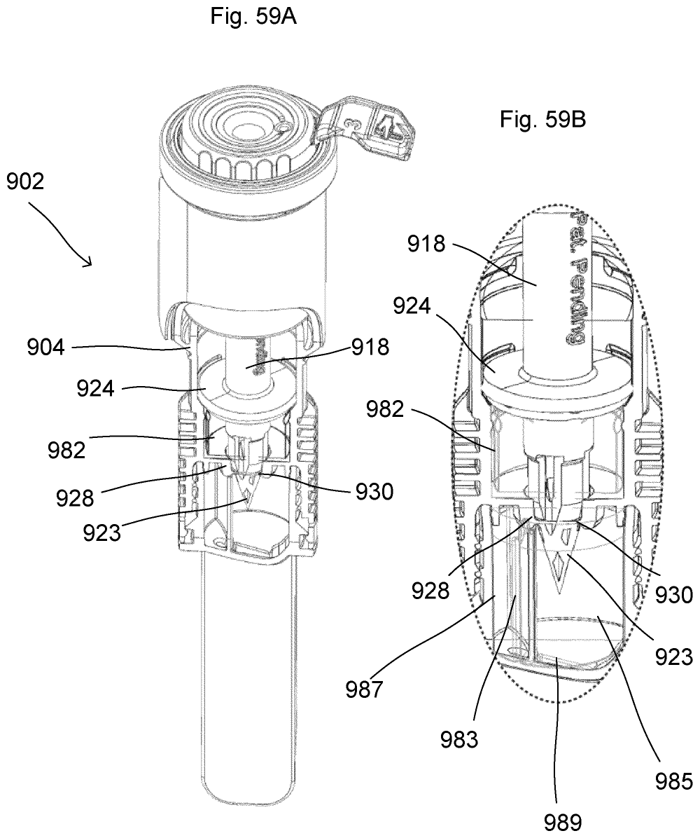

FIG. 59A shows a partial cut-away view through the sample collection device with the sample collection conduit fully advanced into the sample collection chamber; FIG. 59B shows a focussed view in the sample collection chamber with the sample collection conduit in the fully advanced position;

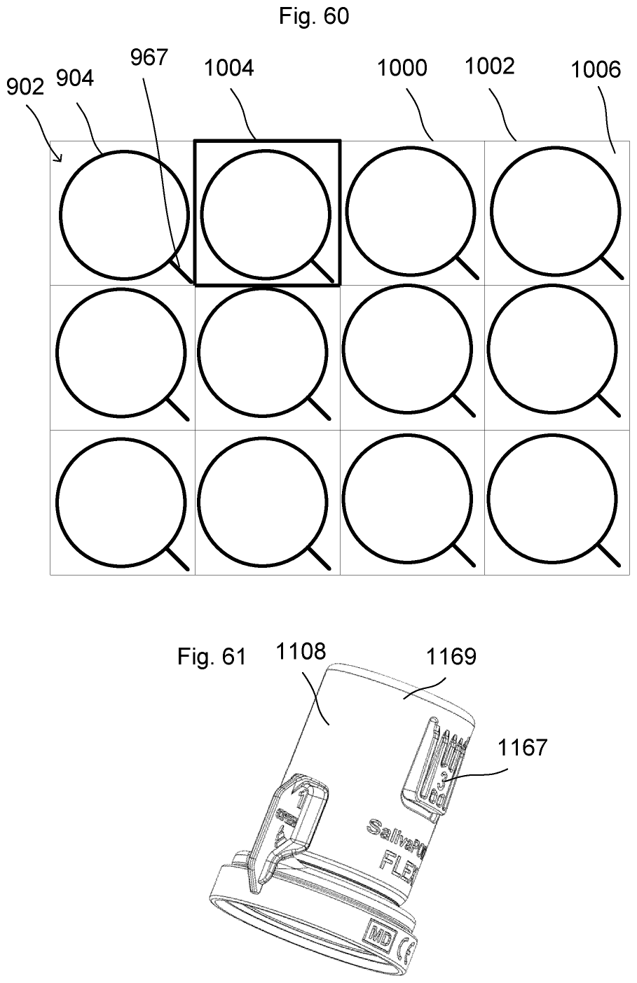

FIG. 60 shows a view from above of a box containing a plurality of sample collection devices; and

FIG. 61 is a perspective view of an inlet cap in accordance with another embodiment of the present invention.

FIG. 1 shows a perspective view of a sample collection device 2, hereinafter “device 2”, in accordance with an embodiment of the present invention. The device 2 comprises a sample collection chamber 4. An outlet cap 3 is connected to a connection arrangement 6 at one end of the sample collection chamber 4 and an inlet cap 8 is attached at the other end thereof. The inlet cap 8 is connected to an inlet cap tamper element in the form of a connection ring 10. The connection ring 10 extends around the sample collection chamber 4 and is retained around the sample collection chamber 4 by a plurality of lugs 12. The device 2 further comprises a release member 16 which will be described in more detail in relation to later FIGURES. The device 2 is depicted in FIG. 1 in a configuration whereby the device 2 has not yet been used to provide a fluid sample, i.e. where the inlet cap 8 is attached to the device 2 such that the connection between the inlet cap 8 and the connection ring 10 has not been broken, and with the outlet cap 3 pre-attached.

FIG. 2 shows a cross-sectional view through the device 2 shown in FIG. 1. As shown in this Figure, the device 2 also comprises a sample collection conduit 18, hereinafter “conduit 18”. The conduit 18 extends out through an opening 19 of the sample collection chamber 4. In the configuration shown in FIG. 2, the inlet cap 8 surrounds the conduit 18. This may help to ensure that the conduit 18 remains sterile before use. The conduit 18 comprises a conduit inlet 20, for receiving a fluid sample, and a conduit outlet 22 which allows the fluid sample to pass out of the conduit 18 into the sample collection chamber 4. In the embodiment depicted, the conduit outlet 22 comprises a pointed tip 23, the purpose of which will be described further below. A plunger 24 and further plunger 26 are operatively linked to the conduit 18 such that movement of the conduit 18 results in corresponding movement of the plunger 24 and further plunger 26. The plunger 24 and further plunger 26 may be integrally formed with the conduit 18, or be separate components which are acted upon by the conduit 18.

At the base, i.e. the second end, of the sample collection chamber 4 is a chamber outlet 28 which is sealed by a seal 30. The seal 30 prevents fluid from passing out of the chamber outlet 28 until the seal is broken, for example by the pointed tip 23 of the conduit 18. The sample collection chamber 4 comprises a first sub-chamber 32 between the plunger 24 and the chamber outlet 28, a second sub-chamber 34 between the plunger 24 and further plunger 26, and a third sub-chamber 36 between the further plunger 26 and the inlet cap 8. The relative positions and volumes of the first, second and third sub-chambers 32, 34, 36 may change as the plungers 24, 26 are advanced into the sample collection chamber 4.

A float member 38 is arranged in the first sub-chamber 32. The float member 38 is ring-shaped and has an opening 39 passing therethrough. The opening 39 may allow fluid to pass through the float member 38 thereby allowing the float member 38 to float on top of a fluid sample. The density of the float member 38 may depend on the type of fluid sample which the device 2 is designed to collect.

The sample collection chamber 4 further comprises a first fluid passageway 40, a second fluid passageway 42, a third fluid passageway 44 and a fourth fluid passageway 46 each of which are formed by recesses in an internal wall 47 of the sample collection chamber 4. It will be appreciated that each of the first, second, third and fourth fluid passageways 40, 42, 44, 46 may comprise a plurality of recesses extending around the internal circumference of the sample collection chamber 4. The fluid passageways 40, 42, 44, 46 provide a means for fluid to bypass the plunger 24 and further plunger 26, and thereby pass between the first, second and third sub-chambers 32, 34, 36. The fluid passageways 40, 42, 44, 46, plunger 24 and further plunger 26 are configured such that when the plunger 24 or further plunger 26 is adjacent the fluid passageway 40, 42, 44, 46, fluid is able to pass through the fluid passageways 40, 42, 44, 46 from one chamber to another. Whereas, when the plunger 24 or second plunger 26 is not fully adjacent one of the fluid passageways 40, 42, 44, 46, the respective fluid passageway 40, 42, 44, 46 will be closed and thus fluid will not be able to pass between the sub-chambers 32, 34, 36. The fluid passageways 40, 42, 44, 46 will be described in further detail below with respect to FIG. 3.

The inlet cap 8 comprises a first circumferential recess 48 on a lower end 49 of the cap 8 and a second circumferential recess 50 extending around a central portion 51 of the cap 50. The first and second circumferential recesses 48, 50 are shaped to receive a locking member 52. The first and second circumferential recesses 48, 50 and the locking member 52 together form part of a restriction arrangement which acts to restrict movement of the inlet cap 8. As the inlet cap 8 is advanced towards the sample collection chamber 4, the locking member 52 will extend into one of the first or second circumferential recesses 48, 50, depending on the position of the inlet cap 8, and hold the cap 8 in position relative to the sample collection chamber 4. The inlet cap 8 further comprises a conduit seal 53 which is arranged to seal the conduit inlet 20 when the conduit seal 53 comes into contact with the conduit inlet 20. The conduit seal 53 may seal the conduit inlet 20 by an interference fit there between.

The device 2 comprises an overspill chamber 54 which is formed between an inner wall portion 56 and an outer wall portion 58 of the sample collection chamber 4. The overspill chamber 54 may collect any fluid which overspills the third sub-chamber 36. The overspill chamber 54 also serves to receive the cap 8 as the cap is advanced relative to the sample collection chamber 4.

As shown in FIG. 2, the release member 16 is coupled to a stop member 60 against which a bottom edge 62 of the cap 8 will come into contact with when the cap 8 is advanced relative to the sample collection chamber 4. The stop member 60 may be moved, through operation of the release member 16, to allow the cap 8 to advance past the stop member 60. The stop member and release member 16 therefore also form part of a restriction arrangement which restricts movement of the inlet cap 8 relative to the sample collection chamber 4.

The connection arrangement 6 which is arranged adjacent the chamber outlet 28, comprises a first connection portion 64 and a second connection portion 66. In the embodiment depicted, the first connection portion 64 comprises an internal thread having a first diameter, and the second connection portion 66 comprises an internal thread having a second, smaller, diameter. The first and second connection portions 64, 66 are axially displaced with one another. Of course the first and second connection portions may have any suitable connection means. For example the first and second connection portions may instead comprise tapered connection which form a friction fit with a component attached thereto. Similarly, further connection portions may also be included.

FIG. 3 shows a cut-away view of the sample collection chamber 4, with the other components of the device 2 removed. This view more clearly shows the first fluid passageway 40, second fluid passageway 42, third fluid passageway 44 and fourth fluid passageway 46 which are formed as recesses in the internal surface 47 of the sample collection chamber 4. The fluid passageways 40, 42, 44, 46 may be formed in any suitable manner during manufacture of the sample collection chamber 4. For example, the fluid passageways 40, 42, 44, 46 may be integrally formed as part of a moulding process. Also visible more clearly in this Figure are the first connection portion 64 and second connection portion 66 which each comprise internal threads.

FIG. 4 shows an isometric view of the outlet cap 3 in isolation. The outlet cap 3 comprises a corresponding connection portion 59 which comprises an external thread. In the embodiment depicted, the corresponding connection portion 59 is dimensioned to engage the first connection portion 64 on the connection arrangement 6 on the sample collection chamber 4.

Use of the device 2 in collecting a fluid sample will now be described with reference to FIGS. 5 to 16. In order to provide a fluid sample into the device, a user may first remove the inlet cap 8. With reference to FIG. 5, the inlet cap 8 may be connected to the connection ring 10 via a plurality of connection tabs 11. In order to separate the inlet cap 8 from the connection ring 10, a user may rotate and/or pull the cap 8 so as to break the connection tabs 11. Once the cap 8 has been separated from the connection ring 10, the cap 8 may be pulled away from the sample collection chamber 4.

FIG. 6 shows the outlet cap 8 separated from the sample collection chamber 4. As depicted, removal of the outlet cap 8 leaves the connection ring 10 in position around sample collection chamber 4. Specifically, the connection ring 10 remains in situ extending around the outermost wall 58 of the sample collection chamber 4. The connection ring 10 is retained in position by the lugs 14 which prevent the connection ring 10 from falling away from the sample collection chamber 4. As shown in FIG. 6, once the outlet cap 8 has been removed, this reveals the conduit 18 into which a fluid sample may be provided. The outlet cap 3 may remain in position at this point to prevent the inadvertent dispensing of fluid out of the chamber outlet 28 (not visible in this Figure).

FIG. 7 shows a cross sectional view of the device 2 before any fluid sample has been provided in order to illustrate the relative positions of the plungers 24, 26 within the sample collection chamber. In the configuration shown in FIG. 7, the conduit 18 is in an initial, i.e. collection, position and the plunger 24 and further plunger 26 are in a corresponding initial position. In the exemplary case whereby the device 2 is used to collect a saliva sample, a user may place their mouth around the conduit inlet 20 and spit or drool their fluid sample into the conduit 18. The sample will pass through the conduit 18, pass out of the conduit outlet 22 and pass into the first sub-chamber 32 of the sample collection chamber 4. With the conduit 18 in the initial position, the plunger 24 is in a corresponding initial position adjacent the first fluid passageway 40 and the further plunger 26 is adjacent the second fluid passageway 42. As a result, both the first fluid passageway 40 and second fluid passageway 42 are open such that fluid can flow from the first sub-chamber 32 to the second sub-chamber 34 and from the second sub-chamber 34 to the third sub-chamber 36. Accordingly, as the fluid sample is provided via the conduit 18, any excess fluid sample or foam may pass out of the first sub-chamber 32 into the second sub-chamber 24. In the exemplary case of a fluid sample in the form of saliva, the saliva typically comprises a liquid portion and a foam portion which comprises a plurality of air bubbles. Advantageously, the foam portion may pass through the first fluid passageway 40. This may increase the liquid portion of the fluid sample which is contained within the first sub-chamber 32. In the initial position, the seal 30 on the chamber outlet 28 is intact and therefore the fluid sample cannot pass out through the chamber outlet 28.

As the fluid sample begins to fill the first sub-chamber 32, the float member 38 will begin to float on the fluid sample and rise within the first sub-chamber 32. The hole 39 in the float member 38 allows the fluid sample to easily pass through the float member 38 thereby allowing the float member 38 to quickly float on top of the fluid sample.

FIG. 8 shows a cross sectional view of the device 2 when the first sub-chamber 32 has been at least partially filled with a fluid sample. As shown in this Figure, the float member 38 has risen within the first sub-chamber 32 with the fluid therein. In the embodiment depicted, the float member 38 has risen to a position whereby it is adjacent a portion of the sample collection chamber 4 which comprises an inner wall 56 and outer wall 58. As a result, the float member 38 may not be visible in this position, whereas the float member 38 may have been visible in the lower portion of the first sub-chamber 32 wherein the sample collection chamber 4 comprises a single-skin outer wall 59. Accordingly, in this embodiment, a user may identify that a sufficient volume of fluid sample has been provided when the float member 38 can no longer be seen. Of course, this is just one way in which the float member 38 may be used and the sample collection chamber may instead be configured such that the float member 38 instead becomes visible when sufficient fluid sample has been provided. In addition or alternatively, the sample collection chamber 4 may be provided with volumetric markings, and alignment of the float member with the volumetric markings may be used to determine the fluid level within the sample collection chamber 4.

Once a sufficient volume of fluid sample has been provided into the device 2, the inlet cap 8 may then be replaced onto the device and pushed into a first intermediate position. This is illustrated in FIG. 9. FIG. 10 is a cross-sectional view of the device 2 with the inlet cap 8 arranged in the first intermediate position as shown in FIG. 9. As the inlet cap 8 is pushed back onto the device 2, the conduit seal 53 will engage with the conduit inlet 20 and seal the conduit inlet 20 via an interference fit. Due to the interaction between the conduit seal 53 and the conduit inlet 20, as the inlet cap 8 is advanced into the first intermediate position shown in FIG. 10, this causes the conduit 18 to move out of the initial position shown in previous Figures, and into a first intermediate position. In the first intermediate position shown in FIG. 10, the plunger 24 and further plunger 26 have been advanced into the sample collection chamber 4 into a corresponding first intermediate position such that they are no longer adjacent the first fluid passageway 40 and second fluid passageway 42. In the position shown, the plunger 24 and further plunger 26 form a sealed relationship with the inner surface 47 of the sample collection chamber 4. As a result, the first sub-chamber 32 and second sub-chamber 34 are sealed and no fluid is able to escape either of these sub-chambers 32, 34.

Additionally, as the inlet cap 8 is advanced into this first intermediate position, the inlet cap 8 extends into the space defined by the overspill chamber 54. The inner surface 47 of the sample collection chamber 4 comprises an expansion portion 49 in which the internal diameter of the inner surface 47 at the expansion portion 49 is less than the external diameter of the further plunger 26. As a result, when the inlet cap 8 is advanced into the first intermediate position, and the conduit 8 is driven into the corresponding first intermediate position, the further plunger 26 will advance into the expansion portion 49. Due to the difference in the diameter of the expansion portion 49 and the further plunger 26, the further plunger 26 causes the expansion portion 49 to expand and press against an inner surface 55 of the inlet cap 8. This may improve the seal between the inlet cap 8 and the sample collection chamber 4, thereby more securely sealing the fluid sample within the sample collection chamber 4. This sealing between the inlet cap 8 and the sample collection chamber 4 may act to seal the third sub-chamber 36. As described previously, the connection ring 10 remains around the sample collection chamber 4 when the inlet cap 8 is removed. When the inlet cap 8 is reattached as described above, the connection ring 10 may reinforce the sample collection chamber 4, and prevent significant deformation thereof. This may therefore act to assist in ensuring a proper seal between the inlet cap 8 and sample collection chamber 4.

When the inlet cap 8 reaches the first intermediate position, the locking member 52 engages with the first circumferential recess 48. This engagement prevents the inlet cap 8 from being removed from the sample collection chamber 4, at least without using excessive force. Additionally, when the inlet cap 8 is in this first position, the bottom edge 62 of the inlet cap abuts against the stop member 60. The stop member 60 therefore acts to prevent the inlet cap 8 from being advanced from this first position, and thereby prevents any further movement of the sample collection conduit. As a result, this may help to prevent a user from inadvertently advancing the inlet cap 8 which may cause the inadvertent dispensing of the fluid sample from the sample collection chamber 4.

With the inlet cap 8 and conduit 18 in the first intermediate position, and with a sufficient fluid volume within in the first sub-chamber 32, the pointed tip 23 of the conduit outlet 38 may pass through the hole 39 in the float member 38 as depicted. The hole 39 on the float member may therefore ensure that the pointed tip 23 of the conduit outlet 22 is not prevented from breaking the seal 30, when required. In the configuration shown in FIG. 10, the pointed tip 23 has not yet reached the seal 30 on the chamber outlet 28, and as a result the fluid sample is contained within the sample collection chamber 4. The outlet cap 3 also remains attached to the connection arrangement 6.

In the arrangement shown in FIG. 10, the fluid sample is securely contained within the device 2, as the first sub-chamber 32, second sub-chamber 34 and third sub-chamber 36 are sealed. The fluid sample may thus be stored within the device 2 and may, for example, be transported within the device 2.

Following collection of the fluid sample using the device 2, it may then be necessary to perform analysis on the fluid sample. The fluid sample may thus need to be dispensed from the device 2 into another component for further analysis. The fluid sample may be dispensed directly out of the chamber outlet 28 onto another component, e.g. part of a testing arrangement. However, FIG. 11 shows an exemplary embodiment whereby a further component in the form of a vial 68 is attached to the connection arrangement 6. FIG. 12 shows a cross-sectional view of the device 2 with the vial 68 attached thereto. As shown in this Figure, the vial 68 comprises an external threaded portion 70 which engages with the first connection portion 64 of the connection arrangement 6. It will be appreciated that a different vial, having an external threaded portion, e.g. a vial having a threaded portion with a smaller diameter, may also be connected to the connection arrangement 6 by engaging with the second connection portion 66. As a result, the device 2 may advantageously be used with a wider variety of further components. As depicted, when the vial 68 is initially attached, the inlet cap 8 remains in the first position such that the seal 30 remains intact and any fluid sample remains within the sample collection chamber 4.

Once the vial 68 has been attached, a user may then begin the process of dispensing the fluid sample from the sample collection chamber 4. As discussed previously, the inlet cap 8 is used to advance the conduit 18, and the plunger 24 within the sample collection chamber 4. Accordingly, in order to dispense the fluid sample, the inlet cap 8 must first be advanced past the first intermediate position shown in FIG. 10. With reference to FIG. 13, the release member 16 which acts to move the stop member 60 may be operated to move the stop member 60 away from the bottom edge 62 of the inlet cap 8. The release member 16 is shown in a released position in FIG. 13. The release member 16 may be arranged to pivot relative to the sample collection chamber 4. The release member 16 may, for example, be pivotally connected to the sample collection chamber 4 by an integrally formed hinge.

FIG. 14 shows a cross-sectional view of the device 2 in the configuration shown in FIG. 13 with the release member 16 in the released position. As shown in this Figure, when the release member 16 is operated, e.g. by rotating the release member 16 relative to the sample collection chamber 4, the stop member 60 is moved away from the bottom edge 62 of the inlet cap 8. Accordingly, the stop member 60 no longer acts to prevent movement of the inlet cap 8. The inlet cap 8 may therefore be advanced further relative to the sample collection chamber 4, and further into the overspill chamber 54. A small amount of force may be required to overcome the engagement between the locking member 52 and the circumferential rim 48. The circumferential rim 48 and locking member 52 may be shaped such that only a small force is required to advance the inlet cap 8 further, but shaped such that a large force is required to pull the inlet cap 8 away from the sample collection chamber 4.

FIG. 15 shows a perspective view of the device 2 with the vial 68 connected and with the inlet cap 8 fully advanced into a final position relative to the sample collection chamber 4, with the release member 16 in a released position. FIG. 16 shows a cross-sectional view through the device 2, with the inlet cap 8 and the conduit 18 in the final position as shown in FIG. 15.

When the inlet cap 8 is in the final position shown in FIG. 16, the conduit 18 is advanced into the final position. As shown, in this final position, the inlet cap 8 is fully advanced into the overspill chamber 54. When in the final position, the locking member 52 engages with the second circumferential rim 50 to hold the inlet cap 8 in position and prevent the inlet cap 8 from being able to be pulled away from the sample collection chamber 4. This may advantageously prevent the device 2 from being reused.

As the inlet cap 8 is advanced from the first intermediate position shown in FIG. 12 towards the final position shown in FIG. 16, this will cause the conduit 18 to advance into the sample collection chamber 4 from the first intermediate position shown in in FIG. 10 into the final position shown in FIG. 16. Movement of the conduit 18 between the first intermediate position and final position will be described with reference to FIG. 16 and FIGS. 17 and 18. As the inlet cap 8 advances the conduit 18 from the first intermediate position towards the final position shown in FIG. 15, the conduit 18 will reach a second intermediate position as shown in FIG. 17. As depicted in this Figure, in the second intermediate position, the plunger 24 is in a corresponding second intermediate position adjacent the third fluid passageway 44 and the further plunger 26 will be adjacent the fourth fluid passageway 46. As the plunger 24 is advanced, whilst adjacent the third fluid passageway 44, fluid within the first sub-chamber 32 will be forced out of the first sub-chamber 32 through the third fluid passageway 44 into the second sub-chamber 34. Simultaneously, if the second sub-chamber is full of fluid, fluid may escape the second sub-chamber 34 into the third sub-chamber 36 via the fourth fluid passageway 46. As shown in FIG. 17, whilst the plunger 24 is adjacent the third fluid passageway 44, the seal 30 on the chamber outlet 28 may remain intact, i.e. the pointed tip 33 of the conduit outlet 22 will not have reached the seal 30.

As the conduit 18 advances further towards the final position, from the second intermediate position, the conduit 18 will reach a third intermediate position, as depicted in FIG. 18. In this third intermediate position, the plunger 24 has reached the bottom 45 of the third fluid passageway 44 and thus the third fluid passageway 44 is closed and fluid is no longer able to escape the first sub-chamber 32. As a result, the maximum volume of fluid that will be dispensed will be the volume defined between the chamber outlet 28 and the plunger 24 when it is aligned with the bottom 45 of the third fluid passageway 44. The third fluid passageway 44 may therefore act as a means for defining a pre-set volume of fluid to be dispensed from the device 2. The fourth fluid passageway 46 is arranged such that the fourth fluid passageway 46 is closed by the further plunger 26 no longer being adjacent the fourth fluid passageway 46, at the same point at which the third fluid passageway 44 is closed.

As depicted in FIG. 18, as the conduit 18 reaches the third intermediate position, the pointed tip 23 of the conduit outlet 23 simultaneously comes into contact with the breakable seal 30, thereby breaking the seal 30 and allowing fluid to pass out of the chamber outlet 28. Breaking the breakable seal 30 simultaneously with the closing of the third fluid passageway 44 may advantageously avoid a pressure build up within the first sub-chamber 32 which may otherwise prevent further movement of the conduit 18.

When the inlet cap 8 advances the conduit 28 from a position whereby the plunger 24 is just past the bottom 45 of the third fluid passageway 44, the pointed tip 30 may then break the seal 30 and the fluid sample may be forced out of the chamber outlet 28 under the pressure exerted by the plunger 24. The fluid sample will pass into the vial 68. The conduit 18 may be thus be advanced from the third intermediate position shown in FIG. 18 to the final position shown in FIG. 16. As the conduit 18 is moved the plunger 26 will expel the fluid sample out of the first chamber 32, through the chamber outlet 28 and into the vial 68. As will be appreciated by those skilled in the art, the conduit seal 53 which seals the conduit inlet 20 may prevent any fluid within the conduit 18 itself from being dispensed due to the formation of a vacuum. This may ensure that only the fluid within the first chamber 32 is dispensed.

FIG. 19 shows a perspective view of a sample collection device 102, hereinafter “device 102”, in accordance with another embodiment of the present invention. Except where explicitly described, the device 102 functions in an identical manner to the device 2 described above. The device 102 comprises an outlet cap 103 which is connected to the connection arrangement 106. An outlet tamper element 172 is connected to a lower end 171 of the connection arrangement 106 and will be described in more detail below.

FIG. 20 shows a perspective view of the outlet cap 103 in isolation. Similarly to the outlet cap 3 described above, the outlet cap 103 comprises a corresponding connection portion 159 in the form of an external thread. The outlet cap further 103 further comprises a circumferential protrusion 174. The circumferential protrusion 174 is shaped to engage the tamper element 172 and the engagement therebetween will be described in further detail below with respect to FIG. 24. The outlet cap 103 further comprises an axial protrusion 176. As depicted in FIG. 21, which shows a cross-sectional view of the device 102, when the outlet cap 103 is attached to the connection arrangement 106, the axial protrusion 176 abuts against the seal 130 on the chamber outlet 128. As a result, when the outlet cap 103 is attached, the seal 130 cannot be broken, and thus no fluid sample is able to pass out through the chamber outlet 128, even if the user were to inadvertently advance the conduit 118 causing the pointed tip 123 to contact the seal 130.

FIG. 22 shows a view focussing on the outlet cap 103, the outlet tamper element 172 and the connection arrangement 106, with the outlet cap 103 connected to the connection arrangement 106. The tamper element 172 is connected to the bottom end 171 of the connection arrangement 106 by a plurality of breakable tabs 178. The tamper element 172 is in the form of a circular ring. The tamper element 172 may be integrally formed with connection arrangement 106 during manufacture. The tamper element 172 comprises saw tooth shaped protrusions 180 which engage with corresponding saw tooth shaped recesses 182 on the connection arrangement 106. The saw toothed shaped protrusions and recesses 180, 182 together act as rotation prevention features. As will be appreciated by those skilled in the art, when the outlet cap 103 is initially screwed into the connection arrangement 106, there will be a frictional force between the outlet cap 103 and the tamper element 172 which will tend to try and rotate the tamper element 172. However, due to the engagement of the protrusions 180 and recesses 182, rotation in the direction in which the outlet cap 103 is rotated during attachment will be prevented. As a result, the breakable tabs 178 will remain in contact. The outlet cap 103 may come pre-attached to the device 102 and thus when the outlet cap 103 is pre-attached the breakable tables 178 will not be broken. It will only be once the outlet cap 103 is later removed that the outlet tabs 178 will become broken, as described in more detail below.

FIG. 23 shows a view of the underside of the connection arrangement 106, with the outlet cap 103 removed. As shown, the tamper element 172 comprises a plurality of radially extending ridges 184. Whilst in the embodiment a plurality of ridges 184 are provided, it will be appreciated that a single ridge may also provide the same function. The ridges 184 comprises a chamfered edge 185 which allows the circumferential rim 174 of the outlet cap 103 to pass over the ridges 185 when the outlet cap 103 is initially attached to the connection portion 106.

FIG. 24 shows a cross-sectional of the device 102, focussing on the outlet cap 103 and connection arrangement 106. As shown in this Figure, the corresponding connection portion 159 of the outlet cap 103 is engaged with the second connection portion 166 such that the outlet cap 103 is attached to the connection arrangement 106. The corresponding connection portion 159 on the outlet cap 103 is in the form of an external thread, and the second connection portion 166 is in the form of an internal thread. When the outlet cap 103 has been connected, the circumferential rim 174 of the outlet cap 103 has been advanced past the ridges 184 on the tamper element 172. When the outlet cap 103 is initially attached to the connection arrangement 106, due to the chamfered edge 185 of the ridges 184 of the tamper element 172, the circumferential rim 174 will force the tamper element 174 to expand in a radial direction, thereby allowing the circumferential rim 174 to pass the ridges 184, and thus connect to the connection arrangement 106.

When it is desired to remove the outlet cap 103, e.g. when a user wishes to expel the fluid sample out of the fluid outlet (not shown on this Figure) the outlet cap 103 may be rotated. As the outlet cap 103 is rotated, the circumferential rim 174 of the outlet cap 103 will engage a top surface 186 of the ridges 184 of the outlet tamper element 172. The top surface 186 is shaped to prevent the circumferential rim 174 from passing over the ridges 184. As a result, further rotation of the outlet cap 103 may drive rotation of the outlet tamper element 172 and/or axial movement of the outlet tamper element 172 away from the connection arrangement 106. As will be appreciated by those skilled in the art, the saw-toothed protrusions 180, and corresponding saw-toothed recesses 182 will prevent rotation in a first direction in which the outlet cap 103 is attached, but permit rotation in the opposite direction in which the outlet cap 103 is removed. As a result, the rotational and/or axial movement of the outlet cap 103 will eventually cause the breakable tabs 178 to break, thereby separating the tamper element 172 from the connection arrangement 106. Further rotation of the outlet cap 103 will cause the outlet cap 103 and tamper element 172 to become completely separated from the connection arrangement.

In the embodiment depicted, when the outlet cap 103 is removed, the outlet tamper element 172 will also be separated from the connection arrangement 106, and will remain attached to the outlet cap 103. As a result, this arrangement may be used to provide an indication that the device 102 has been previously used or tampered with.

FIG. 25 shows a view of the underside of the outlet cap 103. The outlet cap 103 comprises a cavity 186 in which a plurality of radially extending ribs 188 are formed. The cavity 186 and ribs 188 may be used to support another component, e.g. a vial. For example, the outlet cap 103 may be removed from the device 102 and placed upside down on a surface such that the cavity 186 faces upwards. A vial, or other component, may then be inserted into the cavity 188 and be supported, and potentially gripped, by the ribs 188. This may be particularly advantageous for use with vials which have a rounded bottom, and are thus unable to stand vertically without some form of support. When a vial is supported in the outlet cap 103, the outlet cap 103 may advantageously lower the centre of gravity of the vial, thereby reducing the likelihood of the vial tipping over.

As shown in FIG. 25, the ribs 188 have a stepped profile 190 whereby the radial extent of the ribs 188 increases such that the ribs 188 define a first support region 192 and a second support region 194. The second support region 194 has a smaller diameter such that it is capable of supporting smaller components, e.g. vials having a smaller diameter. The outlet cap 103 may therefore be capable of supporting further components, e.g. vials, having different diameters.

FIG. 26 shows a cut-away view of a sample collection chamber 204 in accordance with another embodiment of the present invention. The sample collection chamber 204 is substantially the same as the sample collection chamber 4 described above, except that it further comprises a relief valve 296 in the form of a recess in the internal wall 247 of the sample collection chamber 204. The relief valve 296 is proximal to the chamber outlet 228. Use of the relief valve 296 will now be described with reference to FIG. 27 which shows a cutaway view a sample collection device 202 comprising the sample collection chamber 204 shown in FIG. 26. Except where described below, the sample collection device 204 is otherwise identical to the sample collection device 2 described above. As shown in FIG. 27, when the sample collection conduit 218 approaches the final position, the plunger 224 becomes aligned with the relief valve 296 and as a result, the relief valve 296 is effectively opened. As a result, any air pressure that is built up in the vial 268 may be released through the relief valve 296 and into the second sub-chamber 234. As a result, the pressure in the vial 268 may be reduced, and the vial 268 may be separated from the connection portion 206 more easily. Reducing the pressure may also reduce the risk of the fluid sample from being ejected, e.g. sprayed, out of the vial 268 as it is separated. This may reduce the risk of contamination of the user, other people in the vicinity of the sample collection device 202. Of course further relief valves, e.g. between the second sub-chamber and third sub-chamber 236 may also be provided. In the embodiment depicted, the relief valve 296 is opened when the sample collection conduit 218 reaches the final position. It will be appreciated that the relief valve 296 may be arranged such that it is opened as the sample collection conduit 218 approaches the final position, but then becomes closed when the sample collection conduit 218 actually reaches the final position.

FIG. 28 shows a cross-sectional view of a sample collection device 302 in accordance with another embodiment of the present invention. The sample collection device 302 is identical to the sample collection device 2 discussed above, except for the features explicitly mentioned below. The sample collection device 302 comprises an inlet cap 308 which comprises a first circumferential recess 348 and second circumferential recess 350 which are engaged by the locking member 352 in an identical manner to the embodiments discussed above. Accordingly, the inlet cap 308 may be held in the initial and final positions. The inlet cap 308 further comprises a first intermediate circumferential recess 383 and a second intermediate circumferential recess 385. The first and second intermediate recesses 383, 385 together with the locking member 352 provide a volume control arrangement. The first and second intermediate recesses 383, 385 may be positioned on the inlet cap 208 such that when they are engaged by the locking member 352, a specific predefined volume of fluid has been expelled from the sample collection chamber 304. For example, the first intermediate recess 383 may correspond to 0.5 ml being dispensed and the second intermediate recess 385 may correspond to 0.75 ml of fluid being dispensed. Appropriate marking may be provided on the inlet cap 308 to indicate the corresponding volumes. As will be appreciated, engagement of the locking member 352 and the first and second intermediate recesses 383, 385 may therefore provide a volume control arrangement which controls the amount of fluid dispensed from the sample collection chamber 304.

FIG. 29 shows a cut-away view of a sample collection device 402, and FIG. 30 shows a cross-sectional view of the sample collection device 402, incorporating an alternative volume control arrangement in accordance with another embodiment of the present invention. As shown, the sample collection conduit comprises an adjustable element 443, in the form of a threaded nut, which engages with an outer thread 418B provided on the lower portion 418A of the sample collection conduit 418, below the plunger 424. The position of the adjustable element 443 relative to the sample collection conduit 418 may be adjusted, e.g. during assembly of the device 402, by rotating the adjustable element 443 relative to the sample collection conduit 418. This will adjust the axial position of the adjustable element 443 relative to the sample collection conduit 418. As the sample collection conduit 418 is advanced into the sample collection chamber 404, thereby advancing the plunger 424 so as to dispense fluid from the sample collection chamber 404, the adjustable element 443 will eventually abut against the float member 438 which may abut against a lower internal wall 441 of the sample collection chamber 404. The adjustable element 443 therefore effectively engages the sample collection chamber, via the float member 438. This engagement may prevent further movement of the sample collection conduit 418, and thereby prevent further movement of the plunger 424.

If the adjustable element 443 is positioned closer to the pointed tip 423 less fluid volume will be dispensed, whereas if the adjustable element 443 is positioned closer to the plunger 424, more fluid volume will be dispensed. The adjustable element 443 may therefore provide a volume control arrangement which determines the amount of fluid dispensed from the sample collection device 402. The arrangement of the adjustable element 443 shown in FIGS. 29 and 30 is merely illustrative and any suitable form of adjustable element may be provided and positioned in any appropriate position within the sample collection device in order to control the amount of fluid dispensed.

FIG. 31 shows a perspective view of a sample collection device 502 in accordance with another embodiment of the present invention. The sample collection device 502 functions in a similar manner to the sample collection devices shown in previous Figures, and described above, and thus for conciseness only notable differences will be described. The sample collection device 502 comprises a further component 541 attached to the sample collection chamber 504 at and end at which the chamber outlet thereof (not visible in this Figure) is arranged. The further component 504 is connected to the connection arrangement 506. The further component 541 comprises further chamber 543 which is surrounded by a protective cover 545. A cap 503 is attached to the base of the protective cover 545. The cap 503 may be configured to be suitable for attachment to the connection arrangement 506 of the sample collection device 502 such that the cap 503 may also close the bottom end of the sample collection chamber 504, if required. In addition, or alternatively, the cap 503 may also be configured to be suitable for connection to the further chamber 543, when it is removed from the connection arrangement 506, as shown in FIG. 35 and discussed further below. The further chamber 543 may comprise a medium, e.g. a reagent, arranged therein which interacts with the sample when it is expelled into the further chamber 543. For example, the further chamber 543 may contain a Universal Transfer Medium (UTM®) for preserving the sample.

The protective cover 545 and/or the further chamber 543 may be connected to the connection arrangement 506 via an outlet tamper element 574. The protective cover 543 may also be connected so as to seal the connection arrangement 506. The bottom end 547 of the protective cover 545 may be closed such that protective cover 545 acts to seal the connection arrangement 506, and thus the bottom end of the device 502 in which the collection chamber outlet is arranged. The further component 541 may be pre-attached to the connection arrangement 506.

A reinforcing element in the form of a label 507 is arranged around the sample collection chamber 504. The label 507 may act to reinforce an outer wall of the device 402.

An inlet cap 508 may also be pre-attached in a sealed manner to the sample collection device 502, specifically a body in which the sample collection chamber 504 is provided. The inlet cap 508 is attached to the sample collection chamber 504 via a tamper element 509. The tamper element 509 comprises a tab 549 which may be used to separate the tamper element 509 from the inlet cap 508. The tamper element 509 may advantageously be used to indicate whether the device 504, specifically the inlet cap 508 has been tampered with. If the tamper element 509 is intact, it may be assumed that the device 502, specifically the internal components thereof, are aseptic.

The sealed connection provided by the inlet cap 508 and the protective cover 545 may mean that the sample collection device 502 does not require any further packaging. This particular arrangement may therefore reduce the amount of packaging required which may reduce the cost of the providing the device 502, as well as potentially reducing the amount of waste products which need to be disposed of in relation to the device 502. A restriction member 567 in the form of a radially protruding tab is also arranged on the inlet cap 508. This is discussed in further detail below in relation to further embodiments.

FIG. 32 shows a cross-sectional view through the sample collection device 502. As shown in this view, the further chamber 543 is arranged so as to be in fluid communication with the sample collection chamber 504, via the chamber outlet 528, at least when the seal 530 arranged therebetween is broken. As such, a sample may be expelled from the sample collection chamber 504 into the further chamber 543.

FIG. 33 shows a perspective view of the sample collection device 502, with the cap 503 separated from the protective cover 545. The cap 503 may be connected to the protective cover 545 by a threaded and/or frictional connection. As such, the cap 503 may be separated from the protective cover 545 by rotating and/or pulling the cap 503 relative to the protective cover. The cap 503 may be reconnected to the connection arrangement 506 when the protective cover 545 and further chamber 543 are separated therefrom. The cap 503 may therefore be used to re-seal the sample collection chamber 504 and prevent any remaining sample contained therein from leaking out and contaminating any surface.

FIG. 34 shows a perspective view of the sample collection device 502 with the protective cover 545 removed so as to reveal the further chamber 543. As visible, when the protective cover 545 has been removed, the tamper element 572 remains intact. In this regard, in this embodiment depicted, it is the further chamber 543 which is arranged to break the outlet tamper element 572. In other embodiments, the tamper element 572 may break away from the connection arrangement 506 when the protective cover 545 is removed. The tamper element 572 may function in a similar manner to the tamper element discussed in embodiments above.

Once the sample collection device 502 has been used to collect a fluid sample, and the sample has been expelled into the further chamber 543, the further chamber 543 may then be separated from the connection arrangement 506. Whilst not depicted in FIG. 34, when the further chamber 543 is separated from the connection arrangement 506, the tamper element 572 may at least partially (e.g. fully) break away, thereby indicating that the device 502 has been used and is potentially no longer aseptic.

Once the further chamber 543 has been separated from the rest of the device 502, analysis may then be performed on the sample. However, in some instances it may be desirable to store the sample inside the further chamber 543. Accordingly, in some embodiments, as depicted in FIG. 35, the cap 503 may be attached to the further chamber 543 so as to seal the further chamber 543. An external thread (not visible) on the top of the further chamber 543 may engage with an internal thread (not visible) provided on the cap 503.

FIG. 36 shows a perspective view looking inside the cap 503. As shown, the inside of the cap comprises an internal thread 551. The internal thread 551 is dimensioned so as to engage with a corresponding external thread on the top of the further chamber 543.

FIG. 37 shows a cross-sectional view through the upper end of the sample collection device 502 and FIG. 38 shows a cutaway view through the upper end of the sample collection device 502. With reference to both FIGS. 37 and 38, in this embodiment, the inlet cap 508 seals the opening 519 at the top of the sample collection chamber 504. The inlet cap 508 comprises a circumferential ring 510 which seals against a circumferential sealing element 561. The sealing element 561 is arranged on an upper end of the outer way 558 which extends around the overspill chamber 554. The sealing element 561 is shaped to engage and seal against the circumferential ring 510. In the pre-attached configuration shown in FIGS. 37 and 38, the ring 510 forms an integral part of the inlet cap 508 such that when the ring 510 is sealed against the sealing element 561, the inlet cap 508 seals the opening 519. The ring 510 and sealing element 561 may each be made from a material having a different hardness. In some embodiments, the sealing element 561 is made from a harder material than that of the ring 510 such that the sealing element 561 causes deformation of the ring 510 when it is engaged therewith. This deformation may provide or improve the sealing between the ring 510 and the sealing element 561.

FIG. 39 shows a perspective view of the sample collection device 502 with the label 507 removed. As visible in this Figure, the outer wall portion 558 of the device comprises protrusion in the form of a stop member 560. The stop member 560 protrudes into a space internal of the wall (not visible in this Figure) and is configured to engage a corresponding recess 563 arranged on the inlet cap 508. This may function to hold the cap 508 in an intermediate position as it is advanced relative to the sample collection chamber. However, the Applicant has recognised that the protruding stop member 560 can make manufacture of the device more complicated. Accordingly, in some embodiments, the sample collection device 502 comprises at least one deformation feature 565. In the example depicted, two deformation features 565 are provided for the protruding stop member 560. As shown, the deformation features 565 comprises two slots 565 adjacent the stop member 560. The slots 565 may extend part way through the thickness of the outer wall portion 558 such that the thickness of the outer wall portion 558 adjacent the stop member 560 is thinner than the rest of the outer wall. As a result, during manufacture, a tool, e.g. a mould, may be more easily extracted from the sample collection device 502, as the stop member 560 may more easily deform so as to allow the tool to pass. This is particularly relevant when the stop member 560 extends towards an inner wall (inner wall 566 shown in FIG. 40). The deformation feature(s) 565 may have any suitable form that facilitates deformation of the stop member 560. The deformation feature(s) 565 may be integrally formed with the stop member 560.

FIG. 40 shows a cut-away view through the sample collection device demonstrating how the stop member 560 protrudes into a space, in this case the overspill chamber 554 which is defined between an inner wall portion 556 and the outer wall portion 558. When the inlet cap 508 is used to advance the sample collection conduit 518, the stop member 560 will eventually engage the circumferential recess 563. At this point, the inlet cap 508 may be held in position. In order to further advance the inlet cap 508, a threshold force may need to be applied to the inlet cap 508 in order to push the stop member 560 out of engagement with the recess 563. This threshold force may be defined by the deformation features 565 as it is these features which may at least partially facilitate movement of the stop member 560 relative to the outer wall portion 558.

FIG. 41 shows a cross-sectional view through the sample collection device 502 with the inlet cap 508 partially advanced into the sample collection chamber 504. Before the inlet cap 508 is advanced into this position, the inlet cap 508 may first be removed from the device so as to allow a user to provide a sample. In order to remove the inlet cap 508, the tab 549 may be gripped and pulled so as to remove the tamper element 509 from connection between the inlet cap 508 and the ring 510. The tamper element 509 may be completely detached from the inlet cap 508 (as depicted), or may remain partially attached to the inlet cap 508. In either case, any separation of the tamper element 509 from the inlet cap 508 may be taken to be an indication that the sample collection device 508 has been tampered with in some form.

As depicted in FIG. 41, when the inlet cap 508 is advanced into the sample collection chamber 504, the restriction member 567, which is in the form of a radially extending tab 567, abuts against an upper surface 569 of the ring 510. This acts to limit the amount the inlet cap 508 may be initially moved. The restriction member 567 may abut against any suitable part of the sample collection device 502 and is not limited to abutting against the upper surface 569 of the ring 510. As depicted, the restriction member 567 stops the inlet cap 508 at a position whereby the conduit seal 573 has engaged the sample collection conduit 518 and advanced the sample collection conduit 518 into a position whereby the first sub-chamber 532 is closed, but before the sample collection conduit 518 has penetrated the seal at the chamber outlet (not visible in this Figure). Stopping the sample collection conduit 518 in this position may advantageously prevent accidental ejection of the sample from the sample collection device 502. It may be necessary to remove and/or bend the restriction member 567 before the inlet cap 508 can be advanced further. This is illustrated later with respect to another embodiment.

FIG. 42 shows a cross-sectional view through the sample collection device 502 focussing on the inlet cap 508 thereof. In some embodiments, as depicted, the inlet cap 508 may comprise at least one latch 575 arranged to latch onto the sample collection conduit 518. In the embodiment depicted, the inlet cap 508 comprises three latches 575, although any number may be provided. When the inlet cap 508 is advanced into a position whereby the conduit seal 573 seal the sample collection conduit 518, the latches 575 may engage a circumferential rim 521 arranged at the conduit inlet 520. This engagement may help to ensure that the sample collection conduit 518 is properly sealed by the conduit seal 573.

FIG. 43 shows a perspective view of a sample collection device 602 in accordance with another embodiment of the present invention. The sample collection device 602 is substantially the same as the sample collection device 502 described above, except that the further component 641 attached thereto is different. The further component 641 comprises a further chamber 643 and a protective cover 645. Unlike the embodiment discussed above, a cap is not attached to the bottom of the protective cover 645. Instead a cap 651 is arranged within protective cover 645. Arranging the cap 651 inside the protective cover 645 in this manner may help to ensure that the cap 651 remains aseptic

As depicted in FIG. 44, once a user has provided a sample and the sample has been expelled into the further chamber 643, the protective cover 645 and further chamber 643 may be separated from the connection arrangement 606 of the device 602. In doing so, the outlet tamper element 672 may become separated from the connection arrangement 606, as shown.

As shown in FIG. 45, once the further chamber 643 has been separated from the protective cover 645 and the rest of the device 602, the cap 651, which was initially attached to the bottom end of the further chamber 643, may be removed and attached to the upper end of the further chamber 643 so as to seal the sample within the further chamber 643. The further chamber 643 may then be securely stored and/or transported as required.

FIG. 46A shows another embodiment of a sample collection device 702 which is identical to the sample collection device 502 described above, except that the further component 741 comprises a further chamber 743 but does not comprise any protective cover. In this case, the further chamber 743 may be sealed to the rest of the device. The further chamber 743 may interact with an outlet tamper element 772 so that it is possible to determine that the device is aseptic prior to use. A cap 703 may be attached to the bae of the further chamber 743 and be used to seal the further chamber 743 in a similar manner to the embodiment described above with respect to FIG. 45. In some embodiments, the cap 703 may also be suitable for closing the connection arrangement 706, once the further chamber 743 has been removed. The cap 703 may be attached to the bottom of the further chamber 703 by internal threads (not visible in this Figure). When attached to the bottom of the further chamber 703, as shown, the internal threads and the inside of the cap 703 may remain aseptic. As such, the cap 703 may be used to seal the further chamber 703 when the further chamber 743 is separated from the connection arrangement 706 without risk of the cap 703 contaminating the sample.

FIG. 46B shows a view of the further chamber 743, separated from the rest of the device, with the cap 703 attached to an upper end thereof so as to seal the further chamber 743.

FIG. 47 shows a perspective view of a sample collection device 802 in accordance with another embodiment of the present invention. This embodiment is similar to the sample collection device 602 described above, except that the further component 841 comprises a fluid dispensing device 843, e.g. in the form of a dropper, surrounded by a protective cover 845. As shown in FIG. 48, when a sample 877 has been provided, the sample 877 may be ejected into the fluid dispensing device 843. The fluid dispensing device 843 comprises a removable tab 879 which may be removed in order to allow the sample to be dispensed from the fluid dispensing device 843. The fluid dispensing device 843 may be configured to dispense the sample 877 in drop form. This may allow controlled dispensing of the sample 877. The fluid dispensing device 843 may be used whilst it is attached to the sample collection chamber 804. Equally, the fluid dispensing device 843 may be separated from the sample collection chamber 804 in order to dispense the sample therefrom.

FIG. 49 shows a perspective view of a sample collection device 902 in accordance with another embodiment of the present invention. The sample collection device 902 is identical to the sample collection device 502 described above, except that the further component 941 is different. In this embodiment, the further component is a sample analysis device 941 for analysing the sample. The sample analysis device 941 comprises a sample analysis chamber 943. The sample analysis chamber 943 comprises an analysis strip 995 arranged therein which is configured to indicate the presence of particular markers, e.g. bio markers, within a sample. The sample analysis device may be considered to be a vertical flow assay. Whilst a vertical arrangement is shown, the sample analysis chamber 943 may instead have a horizontal orientation, or be held such that it is horizontal. In this case, the sample analysis device 941 may be considered to be a lateral flow assay or lateral flow device/test.

FIG. 50 shows a cross-sectional view through the sample collection device 902 shown in FIG. 49. As depicted, the sample analysis chamber 943 is connected to the connection arrangement 906 through engagement between an external thread 970 on the sample analysis chamber 943 and the first threaded portion 964 in the connection arrangement 906. In other embodiments, the sample analysis chamber 943, or indeed any other suitable further component, may be integrally provided with the rest of the sample collection device 902, e.g. it may be integrally formed with the sample collection chamber 904.

In some embodiments, as depicted in FIG. 50, the sample collection device 902 may comprise a reagent 981 arranged within the sample collection chamber 904. The reagent 981 may be contained within a sealed container 982 arranged at the bottom of the sample collection chamber 904. Arranging the reagent 981 within a sealed container 982 may prevent the reagent from mixing with the sample until it is desired by a user. The sealed container 982 may be blow formed, the reagent 981 may then be introduced therein, before being sealed. This manufacturing process is commonly known as a blow-fill-seal process. The sealed container 982 may be formed and/or made from an appropriate material such that it can be penetrated by the pointed tip 923 of the sample collection conduit 918. This allows a user to control when the sample and the reagent 981 mix. Whilst a sealed container 982 containing a reagent is disclosed in the context of this embodiment, it will be appreciated that any of the embodiments described above may similarly include a sealed container containing a reagent arranged therein within the sample collection chamber, or indeed any part of the device which may be in fluid communication with the sample collection chamber. Whilst a reagent is described above, any other suitable medium which may interact with the sample may be contained within the sealed container.

FIG. 51 shows a view of the sample analysis device 941 comprising the sample analysis chamber 943 in isolation from the rest of the sample collection device 902. The external thread 970, which engages with the first threaded portion 964 can be seen more clearly in this Figure. An intermediate chamber 982 is arranged at the upper portion of the sample analysis device 941. A partition 983 forms a first intermediate chamber 985 and a second intermediate chamber 987. The partition 983 is arranged such that in use, when the sample is expelled from the sample collection chamber 904, it passes into the first intermediate chamber 985.

FIG. 52 shows a cut-away view through the sample analysis device 941. The first intermediate chamber 985 is in fluid communication with an internal slot 989 (which at least partially defines the sample analysis chamber 943) which receives the analysis strip 985 (not visible in this Figure). The second intermediate chamber 987 comprises an opening 991 which is fluidly coupled to a connection conduit 993 which is coupled to a lower portion of the internal slot 989. As such, in use, when fluid enters the slot 989 at the upper end thereof, air within the internal slot 989 may pass through the connection conduit 993, out through the opening 991 and into the second intermediate chamber 987. As the internal slot 989 may be relatively compact, and may closely match the analysis strip 985 in dimensions, this arrangement may advantageously allow air to escape the bottom of the internal slot 989 thereby allowing the sample to flow down the entire length of the internal slot 989. The Applicant appreciates that this connection conduit 993 is just one way in which air may be allowed to escape the internal slot 989 and envisages any other suitable arrangement for allowing the escape of air. For example, a valve which allows the passage of air, but not liquid, may be arranged at the bottom of the internal slot 989.

FIG. 53 shows a view looking into the first and second intermediate chambers 985, 987 and shows the opening 991 and internal slot 989.

FIGS. 54 to 57 show operation of the sample collection device 902. FIG. 54 shows a perspective view of the sample collection device 902 with the inlet cap 908 pre-attached in a sealed manner as evidenced by the tamper element 909 which is attached and intact. The further component in the form of a sample analysis device 941 comprising a sample analysis chamber 943, containing the analysis strip 995, is also pre-attached. The sample analysis device 941 may also be attached in a sealed manner, such that the device 902 is ready for use. The outlet tamper element 909 is still attached and intact indicating that the sample analysis device 941 has not been tampered with and that the device 902 is still aseptic.

When a user is ready to provide a sample, they may first remove the inlet cap 908 in order to gain access to the sample collection conduit 918. In order to remove the inlet cap 908, the user may pull on the tab 949 to separate the inlet cap 908 from the ring 910, by tearing away the tamper element 909 which connects the inlet cap 908 to the ring 910.

With the inlet cap 908 removed, a user may then provide their sample, e.g. a saliva sample, by placing their mouth around the sample collection conduit 918 and passing their sample through the sample collection conduit 918. FIG. 55 depicts the stage whereby a sample has been provided and which fills the sample collection chamber 904. As shown in this Figure, the float member 938 has floated within the sample collection chamber 904 on top of the sample 977 that has been provided. The floating of the float member 938 to the appropriate level may indicate to the user that a sufficient volume of sample has been provided. At this stage, the reagent 981 is contained within the sealed container 982 and thus the sample 977 has not yet mixed with the reagent 981.

As shown in FIG. 56, once a sample has been provided, a user may then reattach the inlet cap 908 and advance the inlet cap 908 relative to the sample collection chamber 908 to the point at which the restriction member 967, in the form of a radially extending tab, abuts against the upper surface 969 of the ring 910. This prevents further movement of the inlet cap 908. In this position, the inlet cap 908 has advanced the sample collection conduit 918 to a position whereby the pointed tip 923 has penetrated the sealed container 982, thereby allowing the sample 977 and reagent 981 to mix to form a mixture 997. In order to facilitate the mixing, a user may shake the sample collection device 902. The mixture may move within the sample collection chamber 904, and may also flow up the conduit 918 in order to facilitate the mixing.

Once the sample 977 and reagent 981 have been fully mixed forming the mixture 997, the mixture 997 may, if required, be left for a period of time to allow the reagent 981 within the mixture to interact with the sample 977. Once sufficient time has elapsed, the mixture 997 may then be expelled from the sample collection chamber 904 into to sample analysis chamber 943. This is depicted in FIG. 57.

In order to advance the sample collection conduit 918, so as to advance the plunger 924, the restriction member 967 is first moved in order to allow the inlet cap 908 to be advanced further into the sample collection chamber 904. In the embodiment depicted, the restriction member 967 may be bent into a position in which it no longer restricts movement of the inlet cap 908. The inlet cap 908 may then be advanced into the position shown in FIG. 57. In this position, the sample collection conduit 918 has penetrated the seal 930 on the chamber outlet and the plunger 924, which is operatively coupled to the conduit 918, has expelled the mixture 997 through the chamber outlet 928 and into the sample analysis chamber 943. The mixture 997 is absorbed by the analysis strip 995 and may travel along the analysis strip under the force of gravity and/or capillary action. As depicted, the analysis strip 955 comprises a number of indicator lines 999. The indicator lines 999 may be configured to provide any suitable indication within the mixture. For example, the indicator lines 999 may indicate that a valid sample has been provided, i.e. a control line, and another line may indicate the detection of a marker, e.g. antibodies, within the sample which are indicative of a particular virus, e.g. SARS-COV-19. Of course, the sample analysis device 941 may be used to provide an indication of any suitable marker, e.g. biomarker, or material within the sample. Whilst an analysis strip 955 is depicted, the sample analysis chamber 943 may comprise any other suitable means for performing analysis on the sample. For example, the sample analysis chamber 943 may house a liquid solution which changes colour upon detection of a particular marker within the sample.

For completeness, FIG. 58A shows a cut-away view through the sample collection device 902 with the sample collection conduit 918 partially advanced into the sample collection chamber 904, i.e. when in the position shown in FIG. 55. When in this position, as previously described, the pointed tip 923 has penetrated the sealed container 982 such that the contents of the sealed container 982 are free to mix with the sample. FIG. 58B shows a focussed view of the pointed tip 923 penetrating the sealed container 982. As visible in this Figure, the pointed tip 923 penetrates the sealed container 982 before it penetrates the chamber seal 930 on the chamber outlet 928. As such, the sealed container 982 may be penetrated or broken, to allow the mixing of its contents with the sample, before the pointed tip goes on to penetrate the chamber seal 930 at which point the sample may be expelled from the sample collection chamber 904.

FIG. 59A shows a cut-away view of the sample collection device 902 with the cap 908 in the fully advanced position which results in the sample collection conduit 918 being in a fully advanced position within the sample collection chamber 904. This corresponds to the position shown in FIG. 57. FIG. 59B shows a view focussed on the tip 923 penetrating through the chamber seal 930. With reference to both FIGS. 59A and 59B, as the sample collection conduit 918 is advanced further into the sample collection chamber 904, past the position shown in FIGS. 58A and 58B, the pointed tip 930 eventually penetrates the chamber seal 930, thereby allowing fluid to escape the sample collection chamber 904. Once the chamber seal 930 is broken, the plunger 924 expels the sample out of the sample collection chamber 904 via the chamber outlet 928. As can be seen most clearly in FIG. 59B, the partition 989 on the sample collection device 941 is positioned such that any fluid which leaves the chamber outlet 928 will pass into the first intermediate chamber 985 and then into the internal slot 989 in which analysis is performed. The sample does not pass into the second intermediate chamber 987.

Whilst various examples of further components have been described above, it will be appreciated that any number of different further components utilised with any of the above described devices.

FIG. 60 shows a view from above of a packaging box 1000 which houses a plurality of sample collection devices 902. The box 1000 comprises a plurality of square shaped receptacles 1002 each of which receives a separate sample collection device 902. As can be seen in this Figure, the restriction element 967 extends radially away from the body of the sample collection device 902. As shown, the size of each receptacle 1002 substantially matches the size of each sample collection device 902. Specifically, the length of each side of each square receptacle 1002 is only slightly larger than the diameter of each sample collection device 902. In the example depicted, the largest diameter of the device is that of the sample collection chamber 904. As shown, the restriction element 967 extends no further than the perimeter of a square 1004 which has a sides having a length which is just slightly larger than the diameter of the sample collection chamber 904. This particular arrangement may help to provide an efficient means for storing the sample collection devices 902 within the box 1000 as the amount of unoccupied space 1006 may be reduced.

FIG. 61 shows a perspective view of an inlet cap 1108 in accordance with another embodiment of the present invention. The inlet cap 1108 is substantially the same as the inlet cap 508 described above, except that the restriction member 1167 is in a different form. In the embodiment shown in FIG. 61, the restriction member 1167 is in the form of a depressible restriction member 1167 which can be depressed radially inwards. The restriction member 1167 functions in a similar manner to the restriction member 567 described above, in that the restriction member 1167 abuts against a body of the device when the inlet cap 1108 is initially advanced relative to the sample collection chamber. However, the restriction member 1167 is released by depressing the restriction member 1167 radially inwards, rather than bending the restriction member out of abutment with the body of the device. When the restriction member 1167 is depressed radially inwards, the restriction member 1167 may no longer protrude out of the outer wall 1169 of the inlet cap 1108, and the inlet 1108 may then be advanced relative to the device. Whilst the embodiment depicted includes one restriction member 1167, the Applicant has recognised that any number of restriction members may be provided and the inlet cap 1108 can be stopped at a plurality of positions relative to the device. The same applies to any of the restriction members described above.

In any of the embodiments described herein, where a chamber of any type is referred to, the chamber may take any suitable form that is capable of containing a fluid therein. For example, the sample collection chamber may be in the form of a container, e.g. a cylindrical container. In some embodiments, a chamber, e.g. the sample collection chamber, may be integrally formed within a device which has further chambers.

While various different embodiments of sample collection devices each having different features have been described above, it will be appreciated that each of their features may be combined in any suitable combination in line with the scope of the present claims. While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A sample collection device, for collecting a fluid sample, comprising:
a sample collection chamber for collecting a sample therein, wherein the chamber comprises a chamber outlet through which a fluid sample can pass;
a sample collection conduit extending through an opening of sample collection chamber to an outside of the sample collection chamber, wherein the sample collection conduit comprises a conduit inlet for receiving a fluid sample and a conduit outlet for allowing the fluid sample to pass into the sample collection chamber; and
a plunger configured to expel the fluid out of the sample collection chamber through the chamber outlet.

2. The sample collection device of claim 1, wherein the sample collection conduit is moveable relative to the sample collection chamber and wherein the sample collection conduit and plunger are operatively linked such that movement of the sample collection conduit causes movement of the plunger within the sample collection chamber.

3. (canceled)

4. The sample collection device of claim 1, wherein the sample collection chamber comprises a first sub-chamber defined between the plunger and the chamber outlet, wherein the conduit outlet is arranged in the first sub-chamber and wherein the sample collection chamber comprises a first fluid passageway arranged to allow fluid to pass out of the first sub-chamber.

5. The sample collection device of claim 1, wherein the sample collection device comprises a further plunger arranged within the sample collection chamber, and wherein a second sub-chamber is defined between the plunger and further plunger.

6. The sample collection device of claim 5, wherein the further plunger is configured to move together with the plunger, and wherein the sample collection chamber comprises a second fluid passageway arranged to allow fluid to pass out of the second sub-chamber.

7. The sample collection device of claim 1, wherein the plunger is movable to a first intermediate position in which the first fluid passageway is closed.

8. The sample collection device of claim 7, wherein the further plunger is movable into a corresponding first intermediate position whereby the second fluid passageway is also closed.

9-11. (canceled)

12. The sample collection device of claim 1, comprising a volume control arrangement which determines how far the plunger can be advanced into the sample collection chamber.

13. The sample collection device of claim 1, comprising a relief valve arranged to open when the plunger and/or the sample collection conduit approaches a final position in the sample collection chamber.

14. The sample collection device of claim 1, comprising a float member, arranged within the sample collection chamber, configured to float on a fluid sample within the sample collection chamber.

15. (canceled)

16. The sample collection device of claim 1, wherein at least a portion of the sample collection chamber is defined by single skin, at least partially transparent, outer wall such that there is only one wall between the fluid sample collected within the sample collection chamber and an exterior of the sample collection device.

17. The sample collection device of claim 1, comprising an inlet cap arranged to seal the sample collection chamber.

18-25. (canceled)

26. The sample collection device of claim 17, comprising a further plunger, and wherein the inlet cap is arranged to drive the further plunger into the sample collection chamber, and wherein as the further plunger is advanced into the sample collection chamber, the further plunger presses a wall of the sample collection chamber and a wall of the inlet cap together, thereby sealing the opening of the sample collection chamber.

27. The sample collection device of claim 17, comprising a restriction arrangement configured to prevent movement of the inlet cap relative to the sample collection chamber in at least one direction.

28-30. (canceled)

31. The sample collection device of claim 1, comprising an outlet cap, arranged to cover the chamber outlet.

32. (canceled)

33. The sample collection device of claim 31, wherein the chamber outlet is closed by a seal, and wherein at least part of the outlet cap is adjacent the seal such that the seal cannot be broken when the outlet cap is attached to the sample collection device.

34. The sample collection device of claim 1, comprising a further component connected fluidly downstream of the chamber outlet.

35. (canceled)

36. The sample collection device of claim 34, further comprising an outlet tamper element connected to a body of the device or the further component, wherein the outlet tamper element is configured such that when the further component is separated from the chamber outlet, the outlet tamper element breaks away from the body of the device or the further component.

37-43. (canceled)

44. The sample collection device of claim 34, wherein the further component comprises a sample analysis device, configured to analyze the sample.

45-48. (canceled)

49. The sample collection device of claim 1, comprising a connection arrangement arranged fluidly downstream of the chamber outlet, for connecting a further component to the sample collection device.

50-59. (canceled)
